(19) United States
(12) Patent Application Publication
(10) Pub. No.: US 2022/0151994 A1
(43) Pub. Date: May 19, 2022

(54) THERAPEUTIC METHODS AND COMPOSITIONS FOR TREATING LYMPHOMA USING 6,8-BISBENZYLTHIO-OCTANOIC ACID

(71) Applicants: Rafael Pharmaceuticals, Inc., Cranbury, NJ (US); Memorial Sloan Kettering Cancer Center, New York, NY (US); Wake Forest University Health Sciences, Winston-Salem, NC (US)

(72) Inventors: Timothy S. Pardee, Cranbury, NJ (US); Jeffrey Edelson, Cranburry, NJ (US); Ariela Noy, New York, NY (US); Zanetta Lamar, Winston-Salem, NY (US)

(21) Appl. No.: 17/440,229

(22) PCT Filed: Mar. 19, 2020
(86) PCT No.: PCT/US2020/023537

§ 371 Date: Sep. 17, 2021

Related U.S. Application Data
(60) Provisional application No. 62/820,767, filed on Mar. 19, 2019.

(51) Int. Cl. A61K 31/4184; A61K 31/20; A61K 9/00; A61P 35/00

(52) U.S. Cl. CPC A61K 31/4184; A61P 35/00; A61K 9/0019; A61K 31/20

(57) ABSTRACT

The invention provides methods, compositions, and medical kits for treating lymphoma using 6,8-bis-benzylthio-octanoic acid or a pharmaceutically acceptable salt thereof, optionally in combination with a second therapeutic agent.

### CROSS-REFERENCE TO RELATED APPLICATIONS

This application is the national stage application of International (PCT) Patent Application Serial No. PCT/US2020/023537, filed Mar. 19, 2020, which claims the benefit of and priority to U.S. Provisional Patent Application Ser. No. 62/820,767, filed Mar. 19, 2019; the contents of which are hereby incorporated by reference.

### FIELD OF THE INVENTION

The invention provides methods, compositions, and medical kits for treating lymphoma using 6,8-bis-benzylthio-octanoic acid or a pharmaceutically acceptable salt thereof.

### BACKGROUND

Burkitt's Lymphoma/Leukemia (BL) is a highly aggressive hematologic B-cell malignancy classically characterized by the overexpression of c-Myc. Classic types of Burkitt's include endemic, sporadic and immunodeficiency presentations. Due to the rapid proliferation rate of these tumors, the mainstay of treatment includes aggressive chemotherapy and immunotherapy. While intensive combination chemotherapy regimens such as R-EPOCH can provide satisfactory response, some patients will not be cured and the salvage rates in these cases are extremely low (Dunleavy K. et al., “Low-intensity therapy in adults with Burkitt's lymphoma,” New Engl J Med. 2013; 369(20): 1915). NCCN guidelines state that no definitive second line therapies exist.

A highly aggressive subset of diffuse large B cell lymphoma driven by the myc oncogene has been identified. Patients with a dual translocation or triple translocation of myc and the anti-apoptotic gene bcl-2 have an inferior prognosis (Horn H. et al., “MYC status in concert with BCL2 and BCL6 expression predicts outcome in diffuse large B-cell lymphoma,” Blood, 2013 March; 121(12):2253-63). R-CHOP therapy results in inferior outcomes, though R-EPOCH may be suitable (Howlett C. et al., “Front-line, dose-escalated immunochemotherapy is associated with a significant progression-free survival advantage in patients with double-hit lymphomas: a systematic review and meta-analysis,” Br J Haematol., 2015 August; 170(4):504-14). Nevertheless, 20% of patients will have relapsed/refractory disease without viable salvage strategies.

Salvage therapy followed by autologous hematopoietic cell transplant (AuHCT) for patients with relapsed or refractory Hodgkin Lymphoma (HL) or Non-Hodgkin Lymphoma (NHL) is effective only for a subset of patients (Hagberg H. and Gisselbrecht C., “Randomised phase III study of R-ICE versus R-DHAP in relapsed patients with CD20 diffuse large B-cell lymphoma (DLBCL) followed by high-dose therapy and a second randomisation to maintenance treatment with rituximab or not: an update of the CORAL study,” Ann Oncol. 2006; 17 Suppl 4: iv31-32). Relapsed HL may have a more favorable outcome with 71% of patients reportedly without adverse features achieving long-term survival with AuHCT (Majhail N S et al., “Long-term results of autologous stem cell transplantation for primary refractory or relapsed Hodgkin's lymphoma,” Biol Blood Marrow Transplant. 2006; 12:1065-1072). For aggressive NHL (i.e., the high-grade B and T-cell NHL that often present as tumors in the lymph nodes), the outcome in relapsed or refractory disease is less optimistic. One third of transplant eligible patients may obtain long-term disease free survival, but for patients that are transplant ineligible or who recur following transplant, salvage therapy provides few durable remissions.

Thus, a clear unmet medical need exists for additional treatment options for these lymphoma patients. The present invention addresses this need and provides other related advantages.

### SUMMARY

The invention provides methods, compositions, and medical kits for treating a lymphoma using 6,8-bis-benzylthio-octanoic acid or a pharmaceutically acceptable salt thereof. The lymphoma may be, for example, relapsed or refractory. The lymphoma may be, for example, relapsed or refractory Burkitt's lymphoma, double hit diffuse large B-cell lymphoma, relapsed or refractory Hodgkin lymphoma, or relapsed or refractory T-cell non-Hodgkin lymphoma. The 6,8-bis-benzylthio-octanoic acid or a pharmaceutically acceptable salt thereof may be formulated as a pharmaceutical composition, such as a pharmaceutical composition containing an ion pairing agent. The 6,8-bis-benzylthio-octanoic acid or a pharmaceutically acceptable salt thereof may be formulated as a pharmaceutical composition for administration to the patient separate from a pharmaceutical composition containing other agent(s) used in a combination therapy, such as bendamustine or a pharmaceutically acceptable salt thereof.

Accordingly, one aspect of the invention provides a method for treating a lymphoma. The method comprises administering to a patient in need thereof a therapeutically effective amount of 6,8-bis-benzylthio-octanoic acid or a pharmaceutically acceptable salt thereof pursuant to a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered, in order to treat the lymphoma.

Another aspect of the invention provides a medical kit for treating a lymphoma. The medical kit may comprise (i) a therapeutic agent comprising 6,8-bis-benzylthio-octanoic acid or a pharmaceutically acceptable salt thereof, and (ii) instructions for treating lymphoma in a patient using a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered. The instructions may specify, for example, the route of administration for the 6,8-bis-benzylthio-octanoic acid or a pharmaceutically acceptable salt, such as by intravenous administration.

The foregoing aspects of the invention are described in more detail, along with additional embodiments, in the detailed description below.

### DETAILED DESCRIPTION

The invention provides methods, compositions, and medical kits for treating a lymphoma with 6,8-bis-benzylthio-octanoic acid or a pharmaceutically acceptable salt thereof using a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered. The lymphoma may be, for example, characterized as relapsed or refractory. The 6,8-bis-benzylthio-octanoic acid or a pharmaceutically acceptable salt thereof may be formulated as a pharmaceutical composition, such as a pharmaceutical composition containing an ion pairing agent. The 6,8-bis-benzylthio-octanoic acid or a pharmaceutically acceptable salt thereof may be formulated as a pharmaceutical composition for administration to the patient separate from a pharmaceutical composition containing other agent(s) used in a combination therapy, such as bendamustine or a pharmaceutically acceptable salt thereof. The practice of the present invention employs, unless otherwise indicated, conventional techniques of organic chemistry, pharmacology, and biochemistry. Such techniques are explained in the literature, such as “Comprehensive Organic Synthesis” (B. M. Trost & I. Fleming, eds., 1991-1992); which is incorporated by reference. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a,” “an” and “the” as used herein mean “one or more” and include the plural unless the context is inappropriate

The term “6,8-bis-benzylthio-octanoic acid” refers to the compound known as CPI-613, having the chemical structure

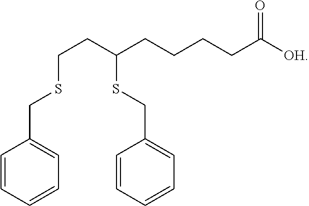 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to organisms to be treated by the methods of the present invention. Such organisms preferably include, but are not limited to, mammals (e.g., murines, simians, equines (horses), bovines (cattle), porcines, canines, felines, and the like). The term “patient” most preferably refers to humans.

As used herein, the term “treating” includes any effect, e.g., lessening, reducing, modulating, ameliorating or eliminating, that results in the improvement of the condition, disease, disorder, and the like, or a symptom thereof.

As used herein, the term “pharmaceutical composition” refers to the combination of an active agent with a carrier, inert or active, making the composition suitable for administration to a human.

The phrase “pharmaceutically acceptable” is employed herein to refer to those compounds, materials, compositions, and/or dosage forms which are, within the scope of sound medical judgment, suitable for use in contact with the tissues of human beings and animals with acceptable toxicity, irritation, allergic response, and other problems or complications commensurate with a reasonable benefit/risk ratio.

As used herein, the term “pharmaceutically acceptable carrier” refers to any of the standard pharmaceutical carriers suitable for use in humans. The compositions also can include stabilizers and preservatives. For examples of carriers, stabilizers, and adjuvants, see e.g., Martin, Remington's Pharmaceutical Sciences, 15th Ed., Mack Publ. Co., Easton, Pa. [1975].

As used herein, the term “pharmaceutically acceptable salt” refers to any salt (e.g., acid or base) of a compound of the present invention which is suitable for administration to a human. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

Further examples of salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Still other examples of salts include anions of the compounds of the present invention compounded with a suitable cation such as Nat, NH₄⁺, and NW₄⁺ (wherein W is a C₁₋₄ alkyl group), and the like. The term “alkyl” is art-recognized, and includes saturated aliphatic groups, including straight-chain alkyl groups and branched-chain alkyl groups.

In certain embodiments, the pharmaceutically acceptable salts are those prepared from the following acids: hydrochloric, hydrobromic, sulfuric, nitric, phosphoric, maleic, acetic, palicylic, p-toluene sulfonic, tartaric, citric, methane sulfonic, formic, malonic, succinic, naphthalene-2-sulfonic, and benzene sulfonic. In certain other embodiments, the pharmaceutically acceptable salts are alkaline metal or alkaline earth salts, such as sodium, potassium or calcium salts of a carboxylic acid group.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Therapeutic Applications

The invention provides a method for treating lymphoma. The method comprises administering to a patient in need thereof a therapeutically effective amount of 6,8-bis-benzylthio-octanoic acid or a pharmaceutically acceptable salt thereof pursuant to a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered, in order to treat the lymphoma. The method may be further characterized according to one or more features described herein.

### Type of Lymphoma

The method may be further characterized according to the severity or type of lymphoma. In certain embodiments, the lymphoma is Stage I lymphoma, in which the cancer is found in one lymph node region or the cancer has invaded one extralymphatic organ or site but not any lymph node regions. In certain embodiments, the lymphoma is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the lymphoma is Stage III lymphoma, in which there is cancer in lymph nodes on both sides of the diaphragm. In certain embodiments, the lymphoma is Stage IV lymphoma, in which the cancer has spread one or more organs beyond the lymph nodes. In certain embodiments, the lymphoma is progressive or refractory. In certain embodiments, the lymphoma is recurrent or relapsed. In certain embodiment, the lymphoma is relapsed or refractory. In certain embodiments, the lymphoma is a T-cell lymphoma. In certain embodiments, the lymphoma is a B-cell lymphoma. In certain embodiments, the lymphoma is previously untreated. In certain embodiments, the patient has not received hematopoietic cell transplant. In certain embodiments, the patient has received hematopoietic cell transplant.

In certain embodiments, the lymphoma is Burkitt's Lymphoma. In certain embodiments, the lymphoma is relapsed or refractory Burkitt's Lymphoma. In certain embodiments, the lymphoma is relapsed or refractory Burkitt's Lymphoma in which the patient has failed at least one previous line of therapy. In certain embodiments, the lymphoma is relapsed or refractory Burkitt's Lymphoma in which the patient has failed prior bone marrow transplant. In certain embodiments, the lymphoma is double hit diffuse large B cell lymphoma. In certain embodiments, the lymphoma is high-grade B cell lymphoma with rearrangements of MYC and BCL2 and/or BCL6 (DHL/THL). In certain embodiments, the lymphoma is Hodgkin lymphoma. In certain embodiments, the lymphoma is non-Hodgkin lymphoma. In certain embodiments, the lymphoma is T-cell non-Hodgkin lymphoma. In certain embodiments, the lymphoma is relapsed or refractory Hodgkin lymphoma. In certain embodiments, the lymphoma is relapsed or refractory non-Hodgkin lymphoma. In certain embodiments, the lymphoma is relapsed or refractory T-cell non-Hodgkin lymphoma. In certain embodiments, the lymphoma is Hodgkin lymphoma in which the patient has not received hematopoietic cell transplant. In certain embodiments, the lymphoma is Hodgkin lymphoma in which the patient has received hematopoietic cell transplant. In certain embodiments, the lymphoma is non-Hodgkin lymphoma in which the patient has not received hematopoietic cell transplant. In certain embodiments, the lymphoma is non-Hodgkin lymphoma in which the patient has received hematopoietic cell transplant. In certain embodiments, the lymphoma is T-cell non-Hodgkin lymphoma in which the patient has not received hematopoietic cell transplant. In certain embodiments, the lymphoma is T-cell non-Hodgkin lymphoma in which the patient has received hematopoietic cell transplant. In certain embodiments, the lymphoma is relapsed or refractory Hodgkin lymphoma in which the patient has not received hematopoietic cell transplant. In certain embodiments, the lymphoma is relapsed or refractory Hodgkin lymphoma in which the patient has received hematopoietic cell transplant. In certain embodiments, the lymphoma is relapsed or refractory non-Hodgkin lymphoma in which the patient has not received hematopoietic cell transplant. In certain embodiments, the lymphoma is relapsed or refractory Hodgkin lymphoma in which the patient has or has not received hematopoietic cell transplant. In certain embodiments, the lymphoma is relapsed or refractory Hodgkin lymphoma in which the patient has failed brentuximab vedotin and a PD-1 inhibitor. In certain embodiments, the lymphoma is relapsed or refractory Hodgkin lymphoma in which the patient has failed brentuximab vedotin and a PD-1 inhibitor and has received hematopoietic cell transplant. In certain embodiments, the lymphoma is relapsed or refractory Hodgkin lymphoma in which the patient has failed brentuximab vedotin and a PD-1 inhibitor and has not received hematopoietic cell transplant. In certain embodiments, the lymphoma is relapsed or refractory non-Hodgkin lymphoma in which the patient has received hematopoietic cell transplant. In certain embodiments, the lymphoma is relapsed or refractory T-cell non-Hodgkin lymphoma in which the patient has not received hematopoietic cell transplant. In certain embodiments, the lymphoma is relapsed or refractory T-cell non-Hodgkin lymphoma in which the patient has received hematopoietic cell transplant. In certain embodiments, the lymphoma is relapsed or refractory T-cell non-Hodgkin lymphoma in which the patient has or has not received hematopoietic cell transplant.

### General Aspects of Administering a Therapeutic Agent to a Patient

Generally, a therapeutic agent is delivered to the patient in a therapeutically effective amount, sufficient to treat the disease or disorder. The treatment may involve one or several administrations on one or more days, and the dosage may be adjusted by the individual practitioner to achieve a desired effect. Preferably, the dosage amount of the agent(s) used should be sufficient to interact primarily with tumor cells, leaving normal cells comparatively unharmed.

The dosage amount may be administered in a single dose or in the form of individual divided doses, such as from one to four or more times per day. Preferably, the daily dosage amount is administered in a single dose. In the event that the response in a subject is insufficient at a certain dose, even higher doses (or effective higher doses by a different, more localized delivery route) may be employed to the extent of patient tolerance.

For combination therapy, components in a combination therapy may be administered in a particular order and/or on the same or different days according to a treatment cycle. For example, in certain embodiments, at least one dose of the 6,8-bis-benzylthio-octanoic acid or a pharmaceutically acceptable salt thereof is administered to the patient prior to administering the second therapeutic agent, such as on an earlier day in a treatment cycle. In certain other embodiments, active components of the combination therapy may be administered on the same day of a treatment cycle, for example being co-administered simultaneously. In certain embodiments, treatment cycles may be repeated one or more times in order to maximize benefit to the patient.

### 6,8-Bis-Benzylthio-Octanoic Acid or Pharmaceutically Acceptable Salt Thereof

In certain embodiments, the therapeutic agent is 6,8-bis-benzylthio-octanoic acid. In certain other embodiments, the therapeutic agent is a salt of 6,8-bis-benzylthio-octanoic acid. In certain embodiments, the therapeutic agent is 6,8-bis-benzylthio-octanoic acid in the form of an ion pair with triethanolamine. In certain embodiments, the therapeutic agent is a triethanolamine salt of 6,8-bis-benzylthio-octanoic acid.

The 6,8-bis-benzylthio-octanoic acid or pharmaceutically acceptable salt thereof may be formulated in a pharmaceutical composition comprising a pharmaceutically acceptable carrier. In certain embodiments, the pharmaceutical composition comprises 6,8-bis-benzylthio-octanoic acid and a pharmaceutically acceptable carrier. In certain embodiments, the pharmaceutical composition comprises an ion pair of 6,8-bis-benzylthio-octanoic acid and a pharmaceutically acceptable carrier. In certain embodiments, the pharmaceutical composition comprises a pharmaceutically acceptable salt of 6,8-bis-benzylthio-octanoic acid and a pharmaceutically acceptable carrier. In certain embodiments, the pharmaceutical composition comprises 6,8-bis-benzylthio-octanoic acid and triethanolamine. In certain embodiments, the pharmaceutical composition comprises 6,8-bis-benzylthio-octanoic acid in the form of an ion pair with triethanolamine. In certain embodiments, the pharmaceutical composition further comprises dextrose and water.

In certain embodiments, the pharmaceutical composition comprises triethanolamine and 6,8-bis-benzylthio-octanoic acid in a mole ratio of triethanolamine to 6,8-bis-benzylthio-octanoic acid of about 10:1 to about 1:10. In certain embodiments, the mole ratio of triethanolamine to 6,8-bis-benzylthio-octanoic acid is about 10:1 to about 5:1. In certain embodiments, the mole ratio of triethanolamine to 6,8-bis-benzylthio-octanoic acid is about 8:1. In certain embodiments, the pharmaceutical composition comprises a 50 mg/mL solution of 6,8-bis-benzylthio-octanoic acid in 1M aqueous triethanolamine. In certain embodiments, the pharmaceutical composition comprises a solution of 6,8-bis-benzylthio-octanoic acid in 1M aqueous triethanolamine diluted from 50 mg/mL to as low as 4 mg/mL with sterile aqueous 5% dextrose for injection (D5W).

Exemplary ion pairing agents that may be used include, for example, a tertiary amine (such as triethanolamine), other amines such as diethanolamine, monoethanolamine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

### Exemplary Route of Administration

The therapeutic method may be further characterized according to the route of administration. For example, in certain embodiments, the 6,8-bis-benzylthio-octanoic acid or pharmaceutically acceptable salt thereof is administered intravenously to the patient. In certain embodiments, the 6,8-bis-benzylthio-octanoic acid or pharmaceutically acceptable salt thereof is administered as an IV infusion over two hours via a central venous catheter.

### Exemplary Dosing Amounts & Regimens

The therapeutic method may be further characterized according to the dose of the 6,8-bis-benzylthio-octanoic acid or pharmaceutically acceptable salt thereof administered to the patient. As used herein, the recited dose refers to the amount of 6,8-bis-benzylthio-octanoic acid administered, such that if a pharmaceutically acceptable salt of higher molecular weight is administered instead of the free acid, the dose of the salt is proportionally higher to provide the recited dose of 6,8-bis-benzylthio-octanoic acid. In certain embodiments, the 6,8-bis-benzylthio-octanoic acid or pharmaceutically acceptable salt thereof is administered at a dosage of about 3,000 mg/m² or less on any day it is administered to the patient. In certain embodiments, the 6,8-bis-benzylthio-octanoic acid or pharmaceutically acceptable salt thereof is administered at a dosage of about 3,000 mg/m² on any day it is administered to the patient. In certain embodiments, the 6,8-bis-benzylthio-octanoic acid or pharmaceutically acceptable salt thereof is administered at a dosage of about 2,750 mg/m² or less on any day it is administered to the patient. In certain embodiments, the 6,8-bis-benzylthio-octanoic acid or pharmaceutically acceptable salt thereof is administered at a dosage of about 2,750 mg/m² on any day it is administered to the patient. In certain embodiments, the 6,8-bis-benzylthio-octanoic acid or pharmaceutically acceptable salt thereof is administered at a dosage of about 2,500 mg/m² or less on any day it is administered to the patient. In certain embodiments, the 6,8-bis-benzylthio-octanoic acid or pharmaceutically acceptable salt thereof is administered at a dosage of about 2,500 mg/m² on any day it is administered to the patient. In certain embodiments, the 6,8-bis-benzylthio-octanoic acid or pharmaceutically acceptable salt thereof is administered at a dosage of about 2,250 mg/m² or less on any day it is administered to the patient. In certain embodiments, the 6,8-bis-benzylthio-octanoic acid or pharmaceutically acceptable salt thereof is administered at a dosage of about 2,250 mg/m² on any day it is administered to the patient. In certain embodiments, the 6,8-bis-benzylthio-octanoic acid or pharmaceutically acceptable salt thereof is administered at a dosage of about 2,000 mg/m² or less on any day it is administered to the patient. In certain embodiments, the 6,8-bis-benzylthio-octanoic acid or pharmaceutically acceptable salt thereof is administered at a dosage of about 2,000 mg/m² on any day it is administered to the patient. In certain embodiments, the 6,8-bis-benzylthio-octanoic acid or pharmaceutically acceptable salt thereof is administered at a dosage of about 1,750 mg/m² or less on any day it is administered to the patient. In certain embodiments, the 6,8-bis-benzylthio-octanoic acid or pharmaceutically acceptable salt thereof is administered at a dosage of about 1,750 mg/m² on any day it is administered to the patient. In certain embodiments, the 6,8-bis-benzylthio-octanoic acid or pharmaceutically acceptable salt thereof is administered at a dosage of about 1,500 mg/m² or less on any day it is administered to the patient. In certain embodiments, the 6,8-bis-benzylthio-octanoic acid or pharmaceutically acceptable salt thereof is administered at a dosage of about 1,500 mg/m² on any day it is administered to the patient. In certain embodiments, the 6,8-bis-benzylthio-octanoic acid or pharmaceutically acceptable salt thereof is administered at a dosage of about 1,250 mg/m² or less on any day it is administered to the patient. In certain embodiments, the 6,8-bis-benzylthio-octanoic acid or pharmaceutically acceptable salt thereof is administered at a dosage of about 1,250 mg/m² on any day it is administered to the patient. In certain embodiments, the 6,8-bis-benzylthio-octanoic acid or pharmaceutically acceptable salt thereof is administered at a dosage of about 1,000 mg/m² or less on any day it is administered to the patient. In certain embodiments, the 6,8-bis-benzylthio-octanoic acid or pharmaceutically acceptable salt thereof is administered at a dosage of about 1000 mg/m² on any day it is administered to the patient. In certain embodiments, the 6,8-bis-benzylthio-octanoic acid or pharmaceutically acceptable salt thereof is administered at a dosage of about 750 mg/m² or less on any day it is administered to the patient. In certain embodiments, the 6,8-bis-benzylthio-octanoic acid or pharmaceutically acceptable salt thereof is administered at a dosage of about 750 mg/m² on any day it is administered to the patient. In certain embodiments, the 6,8-bis-benzylthio-octanoic acid or pharmaceutically acceptable salt thereof is administered at a dosage of about 500 mg/m² on any day it is administered to the patient.

The daily dose of 6,8-bis-benzylthio-octanoic acid or pharmaceutically acceptable salt thereof may vary based on the particular lymphoma being treated. For example, any of the above daily doses may be suitable for treating classic Hodgkin lymphoma or T-cell non-Hodgkin lymphoma, including classic Hodgkin lymphoma or T-cell non-Hodgkin lymphoma that is relapsed or refractory. When treating relapsed or refractory Burkitt's lymphoma the 6,8-bis-benzylthio-octanoic acid or pharmaceutically acceptable salt thereof is preferably administered at a dosage of about 2,500 mg/m² on any day it is administered to the patient. When treating double hit diffuse large B cell lymphoma, the 6,8-bis-benzylthio-octanoic acid or pharmaceutically acceptable salt thereof is preferably administered at a dosage of about 2,500 mg/m² on any day it is administered to the patient. When treating high-grade B cell lymphoma with rearrangements of MYC and BCL2 and/or BCL6 (DHL/THL) the 6,8-bis-benzylthio-octanoic acid or pharmaceutically acceptable salt thereof is preferably administered at a dosage of about 2,500 mg/m² on any day it is administered to the patient.

In certain embodiments, the therapeutic method may be characterized according to the dosing regimen used for administering the 6,8-bis-benzylthio-octanoic acid or a pharmaceutically acceptable salt thereof to the patient. Accordingly, in certain embodiments, the 6,8-bis-benzylthio-octanoic acid or a pharmaceutically acceptable salt thereof is administered pursuant to a treatment cycle of at least two weeks, wherein during each treatment cycle the 6,8-bis-benzylthio-octanoic acid or a pharmaceutically acceptable salt thereof is administered during the first week but is not administered after the first week. In certain embodiments, the treatment cycle is two weeks. In certain embodiments, the treatment cycle is three weeks. In certain embodiments, the treatment cycle is four weeks. In certain embodiments, the treatment cycle comprises an induction phase and a maintenance phase, wherein the dosing regimen in the induction phase is different from the dosing regimen in the maintenance phase. In certain embodiments, the maintenance phase treatment cycle is repeated at least once. In certain embodiments, the maintenance phase comprises at least 2 cycles. In certain embodiments, the maintenance phase comprises at least 3 cycles. In certain embodiments, the maintenance phase comprises at least 4 cycles. In certain embodiments, the maintenance phase comprises at least 5 cycles. In certain embodiments, the maintenance phase comprises at least 6 cycles. In certain embodiments, the maintenance phase comprises at least 7 cycles. In certain embodiments, the maintenance phase comprises at least 10 cycles.

When treating relapsed or refractory Burkitt's lymphoma the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered on days 1-5 of each cycle. When treating high-grade B cell lymphoma with rearrangements of MYC and BCL2 and/or BCL6 (DHL/THL) the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered on days 1-5 of each cycle. When treating double hit diffuse large B cell lymphoma the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered on days 1-5 of each cycle. In certain embodiments, the maintenance phase treatment cycle is repeated at least once. In certain embodiments, the maintenance phase comprises at least 2 cycles. In certain embodiments, the maintenance phase comprises at least 3 cycles. In certain embodiments, the maintenance phase comprises at least 4 cycles. In certain embodiments, the maintenance phase comprises at least 5 cycles. In certain embodiments, the maintenance phase comprises at least 6 cycles. In certain embodiments, the maintenance phase comprises at least 7 cycles. In certain embodiments, the maintenance phase comprises at least 10 cycles.

When treating relapsed or refractory Burkitt's lymphoma the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered at a daily dosage of about 2,500 mg/m² on days 1-5 of each cycle. When treating high-grade B cell lymphoma with rearrangements of MYC and BCL2 and/or BCL6 (DHL/THL) the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered at a daily dosage of about 2,500 mg/m² on days 1-5 of each cycle. When treating double hit diffuse large B cell lymphoma the 6,8-bis-benzylthio-octanoic acid or pharmaceutically acceptable salt thereof is preferably administered pursuant to a treatment cycle comprising an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered at a daily dosage of about 2,500 mg/m² on days 1-5 of each cycle.

When treating classic Hodgkin lymphoma, such as classic Hodgkin lymphoma that is relapsed or refractory or classic Hodgkin lymphoma that is relapsed or refractory in patients who have failed brentuximab vedotin and a PD-1 inhibitor, the 6,8-bis-benzylthio-octanoic acid or pharmaceutically acceptable salt thereof is preferably administered pursuant to treatment cycles of four weeks, in which the 6,8-bis-benzylthio-octanoic acid or pharmaceutically acceptable salt thereof is administered on days 1-4 of each cycle. When treating relapsed or refractory T-cell non-Hodgkin lymphoma the 6,8-bis-benzylthio-octanoic acid or pharmaceutically acceptable salt thereof is preferably administered pursuant to treatment cycles of four weeks, in which the 6,8-bis-benzylthio-octanoic acid or pharmaceutically acceptable salt thereof is administered on days 1-4 of each cycle.

When a four week treatment cycle is used to treat classic Hodgkin lymphoma, such as classic Hodgkin lymphoma that is relapsed or refractory or classic Hodgkin lymphoma that is relapsed or refractory in patients who have failed brentuximab vedotin and a PD-1 inhibitor, or T-cell non-Hodgkin lymphoma, including T-cell non-Hodgkin lymphoma that is relapsed or refractory, the 6,8-bis-benzylthio-octanoic acid or pharmaceutically acceptable salt thereof may be administered at a daily dosage of about 500 mg/m², 750 mg/m², 1,000 mg/m², 1,250 mg/m², 1,500 mg/m², 1,750 mg/m², 2,000 mg/m², 2,250 mg/m², 2,500 mg/m², 2,750 mg/m², or 3,000 mg/m² on days 1-4 of each cycle. In certain embodiments, the 6,8-bis-benzylthio-octanoic acid or pharmaceutically acceptable salt thereof is administered at a daily dosage of about 2,500 mg/m² or less on days 1-4 of each four week treatment cycle. In certain embodiments, the 6,8-bis-benzylthio-octanoic acid or pharmaceutically acceptable salt thereof is administered at a daily dosage of about 3,000 mg/m² or less on days 1-4 of each cycle.

In certain embodiments, the dosing cycle is repeated at least once. In certain embodiments, the method of the present invention comprises treatment with 5 cycles or more. In certain embodiments, the method of the present invention comprises treatment with 6 cycles or more. In certain embodiments, the method of the present invention comprises treatment with 7 cycles or more. In certain embodiments, the method of the present invention comprises treatment with 8 cycles or more. In certain embodiments, the method of the present invention comprises treatment with 9 cycles or more. In certain embodiments, the method of the present invention comprises treatment with 10 cycles or more.

### Second Therapeutic Agent

In certain embodiments, the method of the present invention further comprises administration of a therapeutically effective amount of a second therapeutic agent. For example, the present invention provides a method for treating a lymphoma, comprising administering to a patient in need thereof a therapeutically effective amount of
a. 6,8-bis-benzylthio-octanoic acid or a pharmaceutically acceptable salt thereof, and
b. a second therapeutic agent;
   pursuant to a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered, in order to treat the lymphoma. In certain embodiments, the second therapeutic agent is chemotherapeutic agent. In certain embodiments, the second therapeutic agent is bendamustine or a pharmaceutically acceptable salt thereof. In certain embodiments, the second therapeutic agent is bendamustine hydrochloride.

### Exemplary Route of Administration for the Second Therapeutic Agent

The therapeutic method may be further characterized according to the route of administration of the second therapeutic agent. For example, in certain embodiments, the second therapeutic agent is administered intravenously to the patient. In certain embodiments, the second therapeutic agent is bendamustine hydrochloride and is administered by IV infusion over 10 minutes.

### Exemplary Dosing Amounts & Regimens for the Second Therapeutic Agent

The therapeutic method may be further characterized according to the dose of the second therapeutic agent administered to the patient. Accordingly, in certain embodiments, the second therapeutic agent is administered to the patient at a dosage ranging from about 50 mg/m² to about 150 mg/m² on any day the second therapeutic agent is administered to the patient. In certain embodiments, the second therapeutic agent is administered to the patient at a dosage ranging from about 70 mg/m² to about 120 mg/m² on any day the second therapeutic agent is administered to the patient. In certain embodiments, the second therapeutic agent is administered to the patient at a dosage ranging from about 80 mg/m² to about 100 mg/m² on any day the second therapeutic agent is administered to the patient. In certain embodiments, the second therapeutic agent is administered to the patient at a dosage of about 90 mg/m² on any day the second therapeutic agent is administered to the patient. As used herein, when a dose of bendamustine or a pharmaceutically acceptable salt thereof is recited, it refers to the amount of bendamustine hydrochloride administered, such that if the free base of lower molecular weight or a pharmaceutically acceptable salt of higher or lower molecular weight is administered instead of bendamustine hydrochloride, the dose of the free base or other salt is proportionally lower or higher to provide a dose equivalent to bendamustine hydrochloride.

The therapeutic method may be further characterized according to the dosing regimen used for administering the second therapeutic agent to the patient. When treating classic Hodgkin lymphoma, such as classic Hodgkin lymphoma that is relapsed or refractory or classic Hodgkin lymphoma that is relapsed or refractory in patients who have failed brentuximab vedotin and a PD-1 inhibitor, or T-cell non-Hodgkin lymphoma, including T-cell non-Hodgkin lymphoma that is relapsed or refractory, the second therapeutic agent is preferably bendamustine or a pharmaceutically acceptable salt thereof. In certain embodiments, the second therapeutic agent is bendamustine hydrochloride. When treating classic Hodgkin lymphoma, such as classic Hodgkin lymphoma that is relapsed or refractory or classic Hodgkin lymphoma that is relapsed or refractory in patients who have failed brentuximab vedotin and a PD-1 inhibitor, or T-cell non-Hodgkin lymphoma, including T-cell non-Hodgkin lymphoma that is relapsed or refractory, the bendamustine or pharmaceutically acceptable salt thereof is preferably administered pursuant to treatment cycles of four weeks, in which the bendamustine or pharmaceutically acceptable salt thereof is administered on days 4 and 5 of each cycle. For example, the present invention provides a method of treating relapsed or refractory classic Hodgkin lymphoma or relapsed or refractory T-cell non-Hodgkin lymphoma, comprising the step of administering to a patient in need thereof a therapeutically effective amount of 6,8-bis-benzylthio-octanoic acid or a pharmaceutically acceptable salt thereof pursuant to a treatment cycle of four weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered, and further comprising the step of administering to the patient bendamustine or a pharmaceutically acceptable salt thereof on days 4 and 5 of each cycle.

When treating classic Hodgkin lymphoma, such as classic Hodgkin lymphoma that is relapsed or refractory or classic Hodgkin lymphoma that is relapsed or refractory in patients who have failed brentuximab vedotin and a PD-1 inhibitor, or T-cell non-Hodgkin lymphoma, including T-cell non-Hodgkin lymphoma that is relapsed or refractory, pursuant to a four week treatment cycle with bendamustine or a pharmaceutically acceptable salt thereof as a second therapeutic agent, the bendamustine or pharmaceutically acceptable salt thereof may be administered at a daily dosage of about 50 mg/m², 60 mg/m², 70 mg/m², 80 mg/m², 85 mg/m², 90 mg/m², 95 mg/m², 100 mg/m², 110 mg/m², 120 mg/m², or 130 mg/m² on days 4 and 5 of each cycle. In certain embodiments, the bendamustine or pharmaceutically acceptable salt thereof is administered at a daily dosage of about 100 mg/m² or less on days 4 and 5 of each four week cycle. In certain embodiments, the bendamustine or pharmaceutically acceptable salt thereof is administered at a daily dosage of about 90 mg/m² or less on days 4 and 5 of each four week cycle. In certain embodiments, the bendamustine or pharmaceutically acceptable salt thereof is administered at a daily dosage of about 90 mg/m² on days 4 and 5 of each four week cycle. In certain embodiments, the bendamustine or pharmaceutically acceptable salt thereof is bendamustine hydrochloride and the bendamustine hydrochloride is administered at a daily dosage of about 100 mg/m² or less on days 4 and 5 of each four week cycle. In certain embodiments, the bendamustine hydrochloride is administered at a daily dosage of about 90 mg/m² or less on days 4 and 5 of each four week cycle. In certain embodiments, the bendamustine hydrochloride is administered at a daily dosage of about 90 mg/m² on days 4 and 5 of each four week cycle.

In certain embodiments, the invention provides a method for treating relapsed or refractory classic Hodgkin lymphoma, comprising administering to a patient in need thereof a therapeutically effective amount of
a. 6,8-bis-benzylthio-octanoic acid or a pharmaceutically acceptable salt thereof, and
b. bendamustine or a pharmaceutically acceptable salt thereof;
   pursuant to a treatment cycle of four weeks, wherein the 6,8-bis-benzylthio-octanoic acid or a pharmaceutically acceptable salt thereof is administered only on days 1, 2, 3, and 4 of each treatment cycle and the bendamustine or pharmaceutically acceptable salt thereof is administered only on days 4 and 5 of each treatment cycle, and the 6,8-bis-benzylthio-octanoic acid or a pharmaceutically acceptable salt thereof is administered at a dose of about 3,000 mg/m² or less on each day that it is administered and the bendamustine or pharmaceutically acceptable salt thereof is administered at a dose of about 90 mg/m² on each day that it is administered, in order to treat the lymphoma. In certain embodiments, the patient has failed brentuximab vedotin and a PD-1 inhibitor. In certain embodiments, the 6,8-bis-benzylthio-octanoic acid or pharmaceutically acceptable salt thereof is administered at a dosage of about 2,750 mg/m² or less each day it is administered to the patient. In certain embodiments, the 6,8-bis-benzylthio-octanoic acid or pharmaceutically acceptable salt thereof is administered at a dosage of about 2,750 mg/m² on each day it is administered to the patient. In certain embodiments, the 6,8-bis-benzylthio-octanoic acid or pharmaceutically acceptable salt thereof is administered at a dosage of about 2,500 mg/m² or less on each day it is administered to the patient. In certain embodiments, the 6,8-bis-benzylthio-octanoic acid or pharmaceutically acceptable salt thereof is administered at a dosage of about 2,500 mg/m² on each day it is administered to the patient. In certain embodiments, the 6,8-bis-benzylthio-octanoic acid or pharmaceutically acceptable salt thereof is administered at a dosage of about 2,250 mg/m² or less on each day it is administered to the patient. In certain embodiments, the 6,8-bis-benzylthio-octanoic acid or pharmaceutically acceptable salt thereof is administered at a dosage of about 2,250 mg/m² on each day it is administered to the patient. In certain embodiments, the 6,8-bis-benzylthio-octanoic acid or pharmaceutically acceptable salt thereof is administered at a dosage of about 2,000 mg/m² or less on each day it is administered to the patient. In certain embodiments, the 6,8-bis-benzylthio-octanoic acid or pharmaceutically acceptable salt thereof is administered at a dosage of about 2,000 mg/m² on each day it is administered to the patient. In certain embodiments, the 6,8-bis-benzylthio-octanoic acid or pharmaceutically acceptable salt thereof is administered at a dosage of about 1,750 mg/m² or less on each day it is administered to the patient. In certain embodiments, the 6,8-bis-benzylthio-octanoic acid or pharmaceutically acceptable salt thereof is administered at a dosage of about 1,750 mg/m² on each day it is administered to the patient. In certain embodiments, the 6,8-bis-benzylthio-octanoic acid or pharmaceutically acceptable salt thereof is administered at a dosage of about 1,500 mg/m² or less on each day it is administered to the patient. In certain embodiments, the 6,8-bis-benzylthio-octanoic acid or pharmaceutically acceptable salt thereof is administered at a dosage of about 1,500 mg/m² on each day it is administered to the patient. In certain embodiments, the 6,8-bis-benzylthio-octanoic acid or pharmaceutically acceptable salt thereof is administered at a dosage of about 1,250 mg/m² or less on each day it is administered to the patient. In certain embodiments, the 6,8-bis-benzylthio-octanoic acid or pharmaceutically acceptable salt thereof is administered at a dosage of about 1,250 mg/m² on each day it is administered to the patient. In certain embodiments, the 6,8-bis-benzylthio-octanoic acid or pharmaceutically acceptable salt thereof is administered at a dosage of about 1,000 mg/m² or less on each day it is administered to the patient. In certain embodiments, the 6,8-bis-benzylthio-octanoic acid or pharmaceutically acceptable salt thereof is administered at a dosage of about 1000 mg/m² on each day it is administered to the patient. In certain embodiments, the 6,8-bis-benzylthio-octanoic acid or pharmaceutically acceptable salt thereof is administered at a dosage of about 750 mg/m² or less on each day it is administered to the patient. In certain embodiments, the 6,8-bis-benzylthio-octanoic acid or pharmaceutically acceptable salt thereof is administered at a dosage of about 750 mg/m² on each day it is administered to the patient. In certain embodiments, the 6,8-bis-benzylthio-octanoic acid or pharmaceutically acceptable salt thereof is administered at a dosage of about 500 mg/m² on each day it is administered to the patient.

In certain embodiments, the invention provides a method for treating relapsed or refractory T-cell non-Hodgkin lymphoma, comprising administering to a patient in need thereof a therapeutically effective amount of
a. 6,8-bis-benzylthio-octanoic acid or a pharmaceutically acceptable salt thereof, and
b. bendamustine or a pharmaceutically acceptable salt thereof;
   pursuant to a treatment cycle of four weeks, wherein the 6,8-bis-benzylthio-octanoic acid or a pharmaceutically acceptable salt thereof is administered only on days 1, 2, 3, and 4 of each treatment cycle and the bendamustine or pharmaceutically acceptable salt thereof is administered only on days 4 and 5 of each treatment cycle, and the 6,8-bis-benzylthio-octanoic acid or a pharmaceutically acceptable salt thereof is administered at a dose of about 3,000 mg/m² or less on each day that it is administered and the bendamustine or pharmaceutically acceptable salt thereof is administered at a dose of about 90 mg/m² on each day that it is administered, in order to treat the lymphoma. In certain embodiments, the 6,8-bis-benzylthio-octanoic acid or pharmaceutically acceptable salt thereof is administered at a dosage of about 2,750 mg/m² or less each day it is administered to the patient. In certain embodiments, the 6,8-bis-benzylthio-octanoic acid or pharmaceutically acceptable salt thereof is administered at a dosage of about 2,750 mg/m² on each day it is administered to the patient. In certain embodiments, the 6,8-bis-benzylthio-octanoic acid or pharmaceutically acceptable salt thereof is administered at a dosage of about 2,500 mg/m² or less on each day it is administered to the patient. In certain embodiments, the 6,8-bis-benzylthio-octanoic acid or pharmaceutically acceptable salt thereof is administered at a dosage of about 2,500 mg/m² on each day it is administered to the patient. In certain embodiments, the 6,8-bis-benzylthio-octanoic acid or pharmaceutically acceptable salt thereof is administered at a dosage of about 2,250 mg/m² or less on each day it is administered to the patient. In certain embodiments, the 6,8-bis-benzylthio-octanoic acid or pharmaceutically acceptable salt thereof is administered at a dosage of about 2,250 mg/m² on each day it is administered to the patient. In certain embodiments, the 6,8-bis-benzylthio-octanoic acid or pharmaceutically acceptable salt thereof is administered at a dosage of about 2,000 mg/m² or less on each day it is administered to the patient. In certain embodiments, the 6,8-bis-benzylthio-octanoic acid or pharmaceutically acceptable salt thereof is administered at a dosage of about 2,000 mg/m² on each day it is administered to the patient. In certain embodiments, the 6,8-bis-benzylthio-octanoic acid or pharmaceutically acceptable salt thereof is administered at a dosage of about 1,750 mg/m² or less on each day it is administered to the patient. In certain embodiments, the 6,8-bis-benzylthio-octanoic acid or pharmaceutically acceptable salt thereof is administered at a dosage of about 1,750 mg/m² on each day it is administered to the patient. In certain embodiments, the 6,8-bis-benzylthio-octanoic acid or pharmaceutically acceptable salt thereof is administered at a dosage of about 1,500 mg/m² or less on each day it is administered to the patient. In certain embodiments, the 6,8-bis-benzylthio-octanoic acid or pharmaceutically acceptable salt thereof is administered at a dosage of about 1,500 mg/m² on each day it is administered to the patient. In certain embodiments, the 6,8-bis-benzylthio-octanoic acid or pharmaceutically acceptable salt thereof is administered at a dosage of about 1,250 mg/m² or less on each day it is administered to the patient. In certain embodiments, the 6,8-bis-benzylthio-octanoic acid or pharmaceutically acceptable salt thereof is administered at a dosage of about 1,250 mg/m² on each day it is administered to the patient. In certain embodiments, the 6,8-bis-benzylthio-octanoic acid or pharmaceutically acceptable salt thereof is administered at a dosage of about 1,000 mg/m² or less on each day it is administered to the patient. In certain embodiments, the 6,8-bis-benzylthio-octanoic acid or pharmaceutically acceptable salt thereof is administered at a dosage of about 1000 mg/m² on each day it is administered to the patient. In certain embodiments, the 6,8-bis-benzylthio-octanoic acid or pharmaceutically acceptable salt thereof is administered at a dosage of about 750 mg/m² or less on each day it is administered to the patient. In certain embodiments, the 6,8-bis-benzylthio-octanoic acid or pharmaceutically acceptable salt thereof is administered at a dosage of about 750 mg/m² on each day it is administered to the patient. In certain embodiments, the 6,8-bis-benzylthio-octanoic acid or pharmaceutically acceptable salt thereof is administered at a dosage of about 500 mg/m² on each day it is administered to the patient.

In certain embodiments, the dosing cycle is repeated at least once. In certain embodiments, the method of the present invention comprises treatment with 5 cycles or more. In certain embodiments, the method of the present invention comprises treatment with 6 cycles or more. In certain embodiments, the method of the present invention comprises treatment with 7 cycles or more. In certain embodiments, the method of the present invention comprises treatment with 8 cycles or more. In certain embodiments, the method of the present invention comprises treatment with 9 cycles or more. In certain embodiments, the method of the present invention comprises treatment with 10 cycles or more.

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II clinical trial of at least 5 patients with relapsed or refractory Burkitt's Lymphoma, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II clinical trial comprises at least 10 patients. More preferably, the phase II clinical trial comprises at least 15 patients. More preferably, the phase II clinical trial comprises 16 patients. Preferably, the method of the present invention provides an overall response rate of at least about 20% in patients with relapsed or refractory Burkitt's Lymphoma. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months in patients with relapsed or refractory Burkitt's Lymphoma. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. Preferably, the phase II clinical trial is conducted according to the procedure set forth in Example 1.

When tested in a phase II clinical trial of at least 5 patients with high grade B-cell lymphoma with rearrangements of MYC and BCL2 and/or BCL6 (double hit lymphoma (DHL) or triple hit lymphoma (THL)), the method of the present invention preferably provides an overall response rate of at least about 10%, a duration of response of at least about 1 month, progression-free survival of at least about 1 month, and/or overall survival of at least about 1 month. Preferably, the phase II clinical trial comprises at least 10 patients. More preferably, the phase II clinical trial comprises at least 15 patients. More preferably, the phase II clinical trial comprises 16 patients. Preferably, the method of the present invention provides an overall response rate of at least about 20% in patients with DHL or THL. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months in patients with DHL or THL. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. Preferably, the phase II clinical trial is conducted according to the procedure set forth in Example 1.

When tested in a clinical trial of at least 3 patients with relapsed or refractory Hodgkin lymphoma, the method of the present invention preferably provides a response rate of at least about 10%, a disease control rate (DCR) of at least about 10%, progression-free survival of at least about 1 month, and/or overall survival of at least about 1 month. In certain embodiments, the patients in the clinical trial have failed brentuximab vedotin and a PD-1 inhibitor. Preferably, the clinical trial comprises at least 5 patients. More preferably, the clinical trial comprises at least 7 patients. More preferably, the clinical trial comprises at least 10 patients. More preferably, the clinical trial comprises at least 12 patients. More preferably, the clinical trial comprises at least 15 patients. More preferably, the clinical trial comprises at least 17 patients. More preferably, the clinical trial comprises 19 patients. Preferably, the method of the present invention provides a response rate and/or DCR of at least about 20% in patients with relapsed or refractory Hodgkin lymphoma. More preferably, the method of the present invention provides a response rate and/or DCR of at least about 30%. More preferably, the method of the present invention provides a response rate and/or DCR of at least about 40%. More preferably, the method of the present invention provides a response rate and/or DCR of at least about 50%. More preferably, the method of the present invention provides a response rate and/or DCR of at least about 60%. More preferably, the method of the present invention provides a response rate and/or DCR of at least about 70%. More preferably, the method of the present invention provides a response rate and/or DCR of at least about 80%. More preferably, the method of the present invention provides a response rate and/or DCR of at least about 90%. Preferably, the method of the present invention provides a PFS and/or OS of at least about 2 months in patients with relapsed or refractory Hodgkin lymphoma. Preferably, the method of the present invention provides a PFS and/or OS of at least about 3 months. Preferably, the method of the present invention provides a PFS and/or OS of at least about 4 months. Preferably, the method of the present invention provides a PFS and/or OS of at least about 5 months. Preferably, the method of the present invention provides a PFS and/or OS of at least about 6 months. Preferably, the method of the present invention provides a PFS and/or OS of at least about 7 months. Preferably, the method of the present invention provides a PFS and/or OS of at least about 8 months. Preferably, the method of the present invention provides a PFS and/or OS of at least about 9 months. Preferably, the method of the present invention provides a PFS and/or OS of at least about 10 months. Preferably, the method of the present invention provides a PFS and/or OS of at least about 11 months. Preferably, the method of the present invention provides a PFS and/or OS of at least about 12 months. Preferably, the method of the present invention provides a PFS and/or OS of at least about 14 months. Preferably, the method of the present invention provides a PFS and/or OS of at least about 16 months. Preferably, the method of the present invention provides a PFS and/or OS of at least about 18 months. Preferably, the method of the present invention provides a PFS and/or OS of at least about 20 months. Preferably, the method of the present invention provides a PFS and/or OS of at least about 24 months. Preferably, the clinical trial is conducted according to the procedure set forth in Example 2.

When tested in a clinical trial of at least 3 patients with relapsed or refractory T-cell non-Hodgkin lymphoma, the method of the present invention preferably provides a response rate of at least about 10%, a disease control rate (DCR) of at least about 10%, progression-free survival of at least about 1 month, and/or overall survival of at least about 1 month. Preferably, the clinical trial comprises at least 5 patients. More preferably, the clinical trial comprises at least 7 patients. More preferably, the clinical trial comprises at least 10 patients. More preferably, the clinical trial comprises at least 12 patients. More preferably, the clinical trial comprises at least 15 patients. More preferably, the clinical trial comprises at least 17 patients. More preferably, the clinical trial comprises 19 patients. Preferably, the method of the present invention provides a response rate and/or DCR of at least about 20% in patients with relapsed or refractory T-cell non-Hodgkin lymphoma. More preferably, the method of the present invention provides a response rate and/or DCR of at least about 30%. More preferably, the method of the present invention provides a response rate and/or DCR of at least about 40%. More preferably, the method of the present invention provides a response rate and/or DCR of at least about 50%. More preferably, the method of the present invention provides a response rate and/or DCR of at least about 60%. More preferably, the method of the present invention provides a response rate and/or DCR of at least about 70%. More preferably, the method of the present invention provides a response rate and/or DCR of at least about 80%. More preferably, the method of the present invention provides a response rate and/or DCR of at least about 90%. Preferably, the method of the present invention provides a PFS and/or OS of at least about 2 months in patients with relapsed or refractory T-cell non-Hodgkin lymphoma. Preferably, the method of the present invention provides a PFS and/or OS of at least about 3 months. Preferably, the method of the present invention provides a PFS and/or OS of at least about 4 months. Preferably, the method of the present invention provides a PFS and/or OS of at least about 5 months. Preferably, the method of the present invention provides a PFS and/or OS of at least about 6 months. Preferably, the method of the present invention provides a PFS and/or OS of at least about 7 months. Preferably, the method of the present invention provides a PFS and/or OS of at least about 8 months. Preferably, the method of the present invention provides a PFS and/or OS of at least about 9 months. Preferably, the method of the present invention provides a PFS and/or OS of at least about 10 months. Preferably, the method of the present invention provides a PFS and/or OS of at least about 11 months. Preferably, the method of the present invention provides a PFS and/or OS of at least about 12 months. Preferably, the method of the present invention provides a PFS and/or OS of at least about 14 months. Preferably, the method of the present invention provides a PFS and/or OS of at least about 16 months. Preferably, the method of the present invention provides a PFS and/or OS of at least about 18 months. Preferably, the method of the present invention provides a PFS and/or OS of at least about 20 months. Preferably, the method of the present invention provides a PFS and/or OS of at least about 24 months. Preferably, the clinical trial is conducted according to the procedure set forth in Example 2.

### Patients for Treatment

The therapeutic methods may be further characterized according to the patient to be treated. Preferably, the patient is a human. In certain embodiments, the patient is an adult human.

### III. Medical Kits

Another aspect of the invention provides medical kits containing a therapeutic agent and/or pharmaceutical composition described herein, along with instructions for using the kits to treat a lymphoma according to the therapeutic applications described herein. In certain embodiments, the medical kit comprises (i) 6,8-bis-benzylthio-octanoic acid or a pharmaceutically acceptable salt thereof, and (ii) instructions for treating a lymphoma in a patient using the 6,8-bis-benzylthio-octanoic acid or a pharmaceutically acceptable salt thereof according to the therapeutic applications described herein. In certain embodiments, the medical kit comprises (i) a first therapeutic agent comprising 6,8-bis-benzylthio-octanoic acid or a pharmaceutically acceptable salt thereof, and (ii) instructions for treating lymphoma in a patient using the first therapeutic agent in combination with (a) a second therapeutic agent comprising bendamustine or a pharmaceutically acceptable salt thereof according to the therapeutic applications described herein.

In certain embodiments, the medical kit comprises (i) 6,8-bis-benzylthio-octanoic acid or a pharmaceutically acceptable salt thereof, and (ii) instructions for treating a lymphoma in a patient in need thereof pursuant to a treatment cycle of at least two weeks, wherein during each treatment cycle the 6,8-bis-benzylthio-octanoic acid or a pharmaceutically acceptable salt thereof is instructed to be administered during the first week but not after the first week, and the 6,8-bis-benzylthio-octanoic acid or a pharmaceutically acceptable salt thereof is instructed to be administered at a dose of about 2,500 mg/m² or less on each day that it is administered, in order to treat the lymphoma.

In certain embodiments, the medical kit comprises (i) 6,8-bis-benzylthio-octanoic acid or a pharmaceutically acceptable salt thereof, and (ii) instructions for treating relapsed or refractory Burkitt's lymphoma in a patient in need thereof pursuant to a treatment regimen comprising two 14-day induction cycles followed by one or more 21-day maintenance cycles, wherein the 6,8-bis-benzylthio-octanoic acid or a pharmaceutically acceptable salt thereof is instructed to be administered as a single daily dose of about 2,500 mg/m² on each of days 1, 2, 3, 4, and 5 of each cycle and not administered on other days of the cycle, in order to treat the relapsed or refractory Burkitt's lymphoma.

In certain embodiments, the medical kit comprises (i) 6,8-bis-benzylthio-octanoic acid or a pharmaceutically acceptable salt thereof, and (ii) instructions for treating high-grade B-cell lymphoma with rearrangements of MYC and BCL2 and/or BCL6 in a patient in need thereof pursuant to a treatment regimen comprising two 14-day induction cycles followed by one or more 21-day maintenance cycles, wherein the 6,8-bis-benzylthio-octanoic acid or a pharmaceutically acceptable salt thereof is instructed to be administered as a single daily dose of about 2,500 mg/m² on each of days 1, 2, 3, 4, and 5 of each cycle and not administered on other days of the cycle, in order to treat the high-grade B-cell lymphoma with rearrangements of MYC and BCL2 and/or BCL6.

In certain embodiments, the medical kit comprises (i) 6,8-bis-benzylthio-octanoic acid or a pharmaceutically acceptable salt thereof, and (ii) instructions for treating relapsed or refractory classic Hodgkin lymphoma in a patient in need thereof pursuant to a treatment cycle of four weeks, wherein the 6,8-bis-benzylthio-octanoic acid or a pharmaceutically acceptable salt thereof is instructed to be administered only on days 1, 2, 3, and 4 of each treatment cycle and bendamustine hydrochloride is instructed to be administered only on days 4 and 5 of each treatment cycle, and the 6,8-bis-benzylthio-octanoic acid or a pharmaceutically acceptable salt thereof is instructed to be administered as a single dose of about 2,500 mg/m² on each day that it is administered and the bendamustine hydrochloride is instructed to be administered as a single dose of about 90 mg/m² on each day that it is administered, in order to treat the relapsed or refractory classic Hodgkin lymphoma.

In certain embodiments, the medical kit comprises (i) 6,8-bis-benzylthio-octanoic acid or a pharmaceutically acceptable salt thereof, and (ii) instructions for treating relapsed or refractory T-cell non-Hodgkin lymphoma in a patient in need thereof pursuant to a treatment cycle of four weeks, wherein the 6,8-bis-benzylthio-octanoic acid or a pharmaceutically acceptable salt thereof is instructed to be administered only on days 1, 2, 3, and 4 of each treatment cycle and bendamustine hydrochloride is instructed to be administered only on days 4 and 5 of each treatment cycle, and the 6,8-bis-benzylthio-octanoic acid or a pharmaceutically acceptable salt thereof is instructed to be administered as a single dose of about 2,500 mg/m² on each day that it is administered and the bendamustine hydrochloride is instructed to be administered as a single dose of about 90 mg/m² on each day that it is administered, in order to treat the relapsed or refractory T-cell non-Hodgkin lymphoma.

### IV. Treatment Methods

Another aspect of the invention provides treatment methods in which a therapeutic agent and/or pharmaceutical composition described herein is provided, along with instructions for using it to treat a lymphoma according to the therapeutic applications described herein. In certain embodiments, the treatment method comprises providing (i) 6,8-bis-benzylthio-octanoic acid or a pharmaceutically acceptable salt thereof, and (ii) instructions for treating a lymphoma in a patient using the 6,8-bis-benzylthio-octanoic acid or a pharmaceutically acceptable salt thereof according to the therapeutic applications described herein. In certain embodiments, the treatment method comprises providing (i) a first therapeutic agent comprising 6,8-bis-benzylthio-octanoic acid or a pharmaceutically acceptable salt thereof, and (ii) instructions for treating lymphoma in a patient using the first therapeutic agent in combination with (a) a second therapeutic agent comprising bendamustine or a pharmaceutically acceptable salt thereof according to the therapeutic applications described herein.

In certain embodiments, the treatment method comprises providing (i) 6,8-bis-benzylthio-octanoic acid or a pharmaceutically acceptable salt thereof, and (ii) instructions for treating a lymphoma in a patient in need thereof pursuant to a treatment cycle of at least two weeks, wherein during each treatment cycle the 6,8-bis-benzylthio-octanoic acid or a pharmaceutically acceptable salt thereof is instructed to be administered during the first week but not after the first week, and the 6,8-bis-benzylthio-octanoic acid or a pharmaceutically acceptable salt thereof is instructed to be administered at a dose of about 2,500 mg/m² or less on each day that it is administered, in order to treat the lymphoma.

In certain embodiments, the treatment method comprises providing (i) 6,8-bis-benzylthio-octanoic acid or a pharmaceutically acceptable salt thereof, and (ii) instructions for treating relapsed or refractory Burkitt's lymphoma in a patient in need thereof pursuant to a regimen comprising two 14-day induction cycles followed by one or more 21-day maintenance cycles, wherein the 6,8-bis-benzylthio-octanoic acid or a pharmaceutically acceptable salt thereof is instructed to be administered as a single daily dose of about 2,500 mg/m² on each of days 1, 2, 3, 4, and 5 of each cycle and not administered on other days of the cycle, in order to treat the relapsed or refractory Burkitt's lymphoma.

In certain embodiments, the treatment method comprises providing (i) 6,8-bis-benzylthio-octanoic acid or a pharmaceutically acceptable salt thereof, and (ii) instructions for treating high-grade B-cell lymphoma with rearrangements of MYC and BCL2 and/or BCL6 in a patient in need thereof pursuant to a regimen comprising two 14-day induction cycles followed by one or more 21-day maintenance cycles, wherein the 6,8-bis-benzylthio-octanoic acid or a pharmaceutically acceptable salt thereof is instructed to be administered as a single daily dose of about 2,500 mg/m² on each of days 1, 2, 3, 4, and 5 of each cycle and not administered on other days of the cycle, in order to treat the high-grade B-cell lymphoma with rearrangements of MYC and BCL2 and/or BCL6.

In certain embodiments, the treatment method comprises providing (i) 6,8-bis-benzylthio-octanoic acid or a pharmaceutically acceptable salt thereof, and (ii) instructions for treating relapsed or refractory classic Hodgkin lymphoma in a patient in need thereof pursuant to a treatment cycle of four weeks, wherein the 6,8-bis-benzylthio-octanoic acid or a pharmaceutically acceptable salt thereof is instructed to be administered only on days 1, 2, 3, and 4 of each treatment cycle and bendamustine hydrochloride is instructed to be administered only on days 4 and 5 of each treatment cycle, and the 6,8-bis-benzylthio-octanoic acid or a pharmaceutically acceptable salt thereof is instructed to be administered as a single dose of about 2,500 mg/m² on each day that it is administered and the bendamustine hydrochloride is instructed to be administered as a single dose of about 90 mg/m² on each day that it is administered, in order to treat the relapsed or refractory classic Hodgkin lymphoma.

In certain embodiments, the treatment method comprises providing (i) 6,8-bis-benzylthio-octanoic acid or a pharmaceutically acceptable salt thereof, and (ii) instructions for treating relapsed or refractory T-cell non-Hodgkin lymphoma in a patient in need thereof pursuant to a treatment cycle of four weeks, wherein the 6,8-bis-benzylthio-octanoic acid or a pharmaceutically acceptable salt thereof is instructed to be administered only on days 1, 2, 3, and 4 of each treatment cycle and bendamustine hydrochloride is instructed to be administered only on days 4 and 5 of each treatment cycle, and the 6,8-bis-benzylthio-octanoic acid or a pharmaceutically acceptable salt thereof is instructed to be administered as a single dose of about 2,500 mg/m² on each day that it is administered and the bendamustine hydrochloride is instructed to be administered as a single dose of about 90 mg/m² on each day that it is administered, in order to treat the relapsed or refractory T-cell non-Hodgkin lymphoma.

### V. Pharmaceutical Compositions

Any suitable pharmaceutical composition of 6,8-bis-benzylthio-octanoic acid or pharmaceutically acceptable salt thereof may be used in the therapeutic applications, medical kits, and treatment methods of the present invention. In certain embodiments, the 6,8-bis-benzylthio-octanoic acid or pharmaceutically acceptable salt thereof is formulated as a 50 mg/mL solution in 1 M (150 mg/mL) aqueous triethanolamine, which is diluted from 50 mg/mL to as low as 4 mg/mL with sterile 5% dextrose for injection (D5W) prior to administration as an IV infusion over two hours via a central venous catheter. Preferably, the 50 mg/mL solution is diluted with D5W to a 6,8-bis-benzylthio-octanoic acid concentration of 12.5 mg/mL.

Any suitable pharmaceutical composition of bendamustine or pharmaceutically acceptable salt thereof may be used. In certain embodiments, bendamustine hydrochloride is formulated as a 25 mg/mL solution in polyethylene glycol 400, which further comprises propylene glycol (0.1 mL/mL) and monothioglycerol (5 mg/mL). The solution is transferred to a 50 mL infusion bag of 0.9% Sodium Chloride Injection or 2.5% Dextrose/0.45% Sodium Chloride Injection or D5W to provide a final concentration of bendamustine hydrochloride of about 1.85-5.6 mg/mL, which is infused intravenously to the patient over a period of about 10 minutes. This formulation of bendamustine hydrochloride is commercially available under the brand name BENDEKA®. In certain embodiments, bendamustine hydrochloride is formulated as a lyophilized powder for reconstitution with Sterile Water for Injection. The reconstituted solution is transferred to a 500 mL infusion bag of 0.9% Sodium Chloride Injection or 2.5% Dextrose/0.45% Sodium Chloride Injection to provide a final concentration of bendamustine hydrochloride of about 0.2-0.6 mg/mL, which is infused intravenously to the patient over a period of 30 minutes or less. This formulation of bendamustine hydrochloride is commercially available under the brand name TREANDA®.

The description above describes multiple aspects and embodiments of the invention, including therapeutic applications, treatment methods, pharmaceutical compositions, and medical kits. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and are not intended to limit the invention.

### Example 1—Treatment of Relapsed or Refractory Burkitt's Lymphoma/Leukemia or Double Hit Diffuse Large B Cell Lymphoma in Human Patients Using 6,8-Bis-Benzylthio-Octanoic Acid

### Study Design

A phase II clinical trial of 6,8-bis-benzylthio-octanoic acid (CPI-613) in patients with relapsed or refractory Burkitt's lymphoma/leukemia (Cohort 1) or high-grade B-cell lymphoma with rearrangements of MYC and BCL2 and/or BCL6 (DHL/THL; Cohort 2). Investigators and subjects are not blinded to the treatment.

The primary objective is to determine the overall response rate of CPI-613 in patients with relapsed or refractory Burkitt's Lymphoma/Leukemia and double hit diffuse large B cell lymphoma analyzed as two separate cohorts. Secondary objectives are (a) to evaluate duration of response, progression-free survival (PFS) and overall survival (OS) when CPI-613 is used in patients with relapsed or refractory Burkitt's Lymphoma/Leukemia (BL) and double hit diffuse large B cell lymphoma (DHL), analyzed separately, and (b) to assess the safety of CPI-613 in patients with relapsed or refractory Burkitt's Lymphoma/Leukemia and double hit diffuse large B cell lymphoma, analyzed separately. An exploratory objective is to correlate primary and secondary outcomes with pre-treatment biomarkers including variances in immunohistochemistry and pretreatment cytokine profiles. These will include PBMCs as a source of germline DNA to examine single nucleotide polymorphisms (SNPs) that predict toxicity or resistance/efficacy and plasma for glycolysis/TCA cycle and other metabolites with similar characteristics.

### Patient Inclusion Criteria

Patients must meet all of the following inclusion criteria before enrollment:
1. Must be ≥18 years of age.
2. Histologic diagnosis of Burkitt's Lymphoma/Leukemia or high-grade B-cell lymphoma with rearrangements of MYC and BCL2 and/or BCL6 confirmed at enrolling institution.
3. Failure of at least one previous line of therapy.
4. Failure after prior bone marrow transplant or ineligible for or opted not to participate in bone marrow transplantation for Burkitt's Lymphoma/Leukemia, or DHL/THL.
5. ECOG Performance Status of ≤3.
6. Measurable disease as defined RECIL criteria (2017) or isolated bone marrow involvement.
7. Patients must have fully recovered from the acute, non-hematological, non-infectious toxicities of any prior treatment with anti-cancer drugs, radiotherapy or other anti-cancer modalities. Patients with persistent, non-hematologic, non-infectious toxicities from prior treatment must have documented resolution to ≤Grade 2.
8. Central venous access available (e.g., portacath, PICC line or equivalent).
9. Laboratory values obtained≤2 weeks prior to enrollment must demonstrate adequate hepatic function, renal function, and coagulation as defined below:
   aspartate aminotransferase (AST/SGOT)≤5×upper normal limit (ULN)
   alanine aminotransferase (ALT/SGPT)≤5×ULN
   Total bilirubin≤1.5×ULN (unless related to hemolysis or Gilbert's syndrome)
   creatinine clearance>=40 mL/min either by 24-hour creatine clearance or calculated from the modified Cockcroft=Gault equation (with the use of ideal body mass [IBM] instead of mass): CRCL=(140−Age)×IBM (kg)×[0.85 if female]/[(72·serum creatinine (mg/dL)]
   International Normalized Ratio (INR) must be <1.5. Due to the occurrence of thrombocytopenia, patients with a coagulopathy should not participate. Patients on anticoagulants should be on short-acting therapy (e.g., low molecular weight heparin) rather than oral anticoagulants.
   Albumin≥2.0 g/dL (or ≥20 g/L)
10. Women of child-bearing potential (i.e., women who are pre-menopausal or not surgically sterile) must use accepted contraceptive methods (abstinence, intrauterine device [IUD], oral contraceptive or double barrier device) during the study, and must have a negative serum or urine pregnancy test within 2 weeks prior to treatment initiation.
11. Females must agree to abstain from breastfeeding during study participation
12. Fertile men must practice effective contraceptive methods during the study unless documentation of infertility exists.
13. Patients must have, or be willing and eligible to undergo placement of, a working central venous access device.

### Patient Exclusion Criteria

Patients eligible for this study must not meet any of the following criteria:
1. Patients that have received a chemotherapy regimen with stem cell support in the previous 3 months.
2. Any medical condition that is clinically unstable despite present therapy (i.e. uncontrolled infection).
3. Platelets<50,000/mm³ unless attributable to marrow involvement by lymphoma (either Burkitt lymphoma or DHL/THL). Note: patients with leukemia/lymphoma in the marrow 25,000-50,000 will be assessed for grade 4 thrombocytopenia unless they have platelet recovery above grade 3. Patients entering with platelets<25,000 will only be assessed for thrombocytopenia related to drug if they recover to grade 3 or higher.
4. Serious medical illness, such as significant cardiac disease (e.g. symptomatic congestive heart failure, unstable angina pectoris, coronary artery disease, myocardial infarction within the past 3 months, uncontrolled cardiac arrhythmia, pericardial disease or New York Heart Association Class III or IV), or severe debilitating pulmonary disease, that would potentially increase patient's risk for toxicity.
5. Patients with active central nervous system (CNS) parenchymal disease. Patients with leptomeningeal disease are allowed as long as the CSF has cleared for more than 4 weeks and the patient is receiving maintenance intrathecal/intraOmmaya therapy.
6. Any active uncontrolled bleeding or bleeding diathesis (e.g., active peptic ulcer disease).
7. Any condition or abnormality which may, in the opinion of the investigator, compromise his or her safety.
8. Life expectancy less than 2 months.
9. Requirement for immediate palliative treatment of any kind including surgery.
10. HIV patients with any of the following: a) uncontrolled HIV infection defined as an HIV viral load>100K copies/mL, b) a documented opportunistic infection within the last 90 days, c) concurrent HIV therapy with zidovudine or any strong CYP3A4 inhibitor (e.g., ritonavir or cobicistat) within the past 2 weeks prior to initiation of CPI-613 treatment.
11. Psychiatric illness or social situation that would limit the patient's ability to tolerate and/or comply with study requirements.
12. Prior allogeneic stem cell transplant within 2 months of study start
   a. Patients with active graft-versus-host-disease are not eligible. Patients receiving immunosuppressive therapy for prevention of graft-versus-host disease are not eligible.

### Pretreatment Evaluation

The following assessments will be done prior to study treatment:

Prior to study treatment:
Histologic diagnosis of Burkitt's Lymphoma/Leukemia or high-grade B-cell lymphoma with rearrangements of MYC and BCL2 and/or BCL6 confirmed at enrolling institution
If available, 5 FFPE tumor slides, unstained and paraffin dipped.

Within 4 weeks prior to Day 1 of study treatment:
Bone marrow biopsy: core biopsy, aspirate and cell markers. If a core biopsy has already been done within the 4-week window without an aspirate and cell markers, it does not need to be repeated. If a marrow is being done, the patient should sign consent first to allow for a research sample (5 cc in EDTA).
PET/CT

Within 2 weeks prior to Day 1 of study treatment:
Screening assessments including:
Record prior medications and treatments
Assessment of concomitant medications
Physical exam and medical history
ECOG performance status
ECG
Laboratory tests including clinical chemistry and hematology
   CBC, comprehensive metabolic panel (includes Na, K, Cl, CO₂, Ca, Total Protein, Albumin, Creatinine, Glucose, BUN, Alkaline Phosphate, Alanine Aminotransferase (ALT), Aspartate Aminotransferase (AST), Total Bilirubin, uric acid, phosphorus, EGFR African American, EGFR Non-African American, Anion Gap
Urine or serum pregnancy test for women of child-bearing potential

### Treatment/Intervention Plan

Administration of CPI-613 is described in the table below. Briefly, the first two treatment cycles are 14-days in duration, with all subsequent cycles being 21-days in duration. CPI-613 (2,500 mg/m²/day) is given on Days 1 through 5 of each treatment cycle. Note that the CPI-613 dose may be modified if toxicity/side effects are observed as discussed below. Study treatment will be provided in outpatient chemotherapy units.

| Treatment Cycle | | | |
|---|---|---|---|
| Cycle | | Day | Administration of CPI-613 (2,500 mg/m²/day) |
| Induction | Cycles 1 and 2 | Days 1-5 | CPI-613 2,500 mg/m² IV infusion over 2 hours |
| | (14-day cycles) | | (+/− 10 mins) via a central venous catheter |
| | | Days 6-14 | No treatment |
| Maintenance | Cycle 3 and | Days 1-5 | CPI-613 2,500 mg/m² IV infusion over 2 hours |
| | beyond | | (+/− 10 mins) via a central venous catheter |
| | (21-day cycles) | Days 6-21 | No treatment |

The amount of CPI-613 at each dose level is based on the BSA of the patient. The BSA values will be calculated based on the height and body weight taken during screening and this BSA value is used throughout the study. This is unless there is a >10% change in the body weight from baseline during the study. At that point, BSA should be revised based on the new body weight and height. The new BSA values will be used from that point on for the remainder of the study, unless there is another >10% change in body weight which will require another revision of the BSA.

Concomitant and prophylactic treatment for drug-related symptoms including diarrhea and nausea are allowed. Supportive treatment may include anti-emetic, anti-diarrhea, anti-pyretic, anti-allergic, anti-hypertensive medications, analgesics, antibiotics, allopurinol, and others such as blood products and bone marrow growth factors. All concomitant medications must be recorded in the electronic database eCRFs. Patients cannot receive any standard or investigational treatment (except CPI-613) for their cancer, or any other investigational drugs for any indications, while on this study.

All patients will receive allopurinol starting 24 hours prior to the initiation of therapy and continued throughout induction cycles 1 and 2. Minimum dose of allopurinol 300 mg p.o. daily. Additional measures such as hospitalization with aggressive IV hydration and/or rasburicase will be at the discretion of the investigator.

### Toxicities/Side Effects

An adverse event (AE) is any untoward medical occurrence in a study subject and does not necessarily have a causal relationship with CPI-613. An AE therefore can be any unfavorable and unintended sign (including laboratory finding), symptom or disease temporally associated with participation in an investigational study, whether or not considered drug-related. In addition to new events, any increase in the severity or frequency of a pre-existing condition that occurs after the subject signs a consent form for participation is considered an AE. This includes any side effect, injury, toxicity, or sensitivity reaction.

Whenever possible, the Common Terminology Criteria for Adverse Events (CTCAE) version 5.0 should be used to define the event and for assessing the severity of AEs. Any events representing a change in the CTCAE Grade need to be reported in CRDB. This includes any change in laboratory values. The CTCAE v. 5.0 can be found on the Cancer Therapy Evaluation Program (CTEP) website at:

https://ctep.cancer.gov/protocolDevelopment/electronic_applications/ctc.htm. For AEs not adequately addressed in the CTCAE, the severity table below may be used.

| Severity | Description |
|---|---|
| GRADE 1 - Mild | Transient or mild discomfort; no limitation in activity; no medical |
| | intervention/therapy required. |
| GRADE 2 - Moderate | Mild to moderate limitation in activity-some assistance may be |
| | needed; no or minimal medical intervention/therapy required. |
| GRADE 3 - Severe | Marked limitation in activity, some assistance usually required; |
| | medical intervention/therapy required, hospitalizations possible. |
| GRADE 4 - Life- | Extreme limitation in activity, significant assistance required; life- |
| threatening | threatening (immediate risk of death); significant medical |
| | intervention/therapy required, hospitalization or hospice care |
| | probable. |
| GRADE 5 - Fatal | Death |

Any condition, laboratory abnormality, or physical finding with an onset date prior to the subject signing consent for study participation is considered to be pre-existing in nature and part of the subject's medical history.

All AEs (e.g., any new event or worsening in severity or frequency of a pre-existing condition or laboratory finding) with an onset date after the subject signs consent for study participation must be promptly documented on the appropriate summary Details of the event must include severity, relationship to study drug, duration, action taken, and outcome. All AEs that are considered related to study procedures must be followed to resolution or stabilization if improvement is not expected.

AEs should be reported from the time the subject signs consent through 30 days post-last study intervention. In addition, the Investigator should report any AE that may occur after this time period that is believed to have a reasonable possibility of being associated with study intervention. If a subject discontinues study prior to receiving any study intervention, AEs must be reported through the end-of-study visit. AEs which completely resolve and then recur should be recorded as a new AE. For subjects who complete the end of study visit less than 30 days following the last study intervention, a follow up of ongoing AEs should be attempted by telephone and documented in the subject's source. AEs continuing at 30 days post-last treatment should have a comment in the source by the Investigator that the event has stabilized or is not expected to improve.

The Principal Investigator is responsible for evaluating all AEs, obtaining supporting documents, and determining that documentation of the event is adequate. Adverse events will be assigned a severity grade using the NCI CTCAE grading scale v5.0 and recorded.

Given the limited data for treating Burkitt lymphoma and other hematological malignancies with CPI-613, the following are the most frequent toxicities seen in prior CPI-613 studies treating other types of cancers. Common side effects of CPI-613 in prior studies include nausea, leukocytopenia, neutrophils (neutropenia), fatigue, and elevated alkaline phosphatase (ALP). Occasional side effects of CPI-613 in prior studies include diarrhea, vomiting, hemoglobin (anemia), abnormal liver function tests (elevated bilirubin, alanine aminotransferase (ALT), elevated aspartate aminotransferase (AST)), hyponatremia, hematuria, hypoalbuminemia, anorexia, dysgeusia, hypocalcemia, flushing fever, thrombocytopenia, injection site reaction, lymphopenia, hyperglycemia, increased creatinine, and hypokalemia. Rare but serious side effects of CPI-613 in prior studies include infections, disseminated intravascular coagulation, elevated troponin, and renal failure.

For treatment interruptions during cycle, the 21-day schedule of each cycle will continue to be followed. Missed doses of CPI-613 are not made up if due to toxicity but can be made up if it is due to scheduling (e.g., transportation or inclement weather). For treatment interruptions that delay the scheduled start of a new cycle, when toxicity has resolved as required to allow the start of a new cycle, the restart day of therapy becomes Day 1 of the next cycle. For toxicities attributed as at least partially related to CPI-613, dose adjustment will be as outlined in the table below. Grade 4 non-hematologic toxicity must be ≤7 days. Patients will be removed from study for any grade 3-4 non-hematologic toxicity occurring on a CPI-613 50% dose reduction.

| | |
|---|---|
| | Supportive Care and Dose |
| Toxicity and Intensity | Adjustment Guidelines |

Nausea, Acute (common)

| | |
|---|---|
| Grade 1 or 2 | Maintain dose and schedule. Rule out other causes. Utilize |
| (If intolerable or persistent | anti-nausea medications including 5-HT3 antagonists. |
| Grade 2 not responsive to | |
| supportive care, follow | |
| guidelines for Grade 3) | |
| Grade 3-4 | Rule out other causes. Utilize anti-nausea medications |
| Grade 4 toxicity | including 5-HT3 antagonists. |
| must be ≤ 7 days | Interrupt CPI-613 dosing until resolved to Grade 1 or baseline |
| | and reduce CPI-613 dose by 50%. |

Diarrhea (common)

| | |
|---|---|
| Grade 1 | Maintain dose and schedule. Rule out other causes including |
| | drug effects. Treat per institutional guidelines with |
| | anti-diarrheals. |
| Grade 2 | Rule out other causes including drug effects. Treat per |
| | institutional guidelines with anti-diarrheals. Interrupt CPI-613 |
| | dosing until resolved to Grade 1 or baseline. |
| | For first occurrence, restart CPI-613 at current dose. |
| | For ≥ second occurrence, reduce dose by 25%. |
| Grade 3 or 4 | Interrupt CPI-613 dosing until resolved to Grade 1 or baseline |
| Grade 4 toxicity must be ≤7 days | and patient is clinically stable. Reduce CPI-613 dose by 50%. |

Renal Failure

| | |
|---|---|
| Grade 1 | Maintain dose and schedule. |
| Grade 2 | Hold dose until resolved to grade 1 or baseline and resume |
| | CPI-613 at a 25% dose reduction. |
| Grade ≥3 | Hold dose until resolved to grade 1 or baseline and resume |
| Grade 4 toxicity must be ≤7 days | CPI-613 at a 50% dose reduction. |

Non-Hematological Adverse Events

| | |
|---|---|
| Grade 1 | Maintain dose and schedule. |
| Grade 2 | Hold dose until resolved to grade 1 or baseline and resume |
| | CPI-613 at a 25% dose reduction. |
| Grade 3 or 4 | Hold dose until resolved to grade 1 or baseline and resume |
| Grade 4 non-hematologic toxicity | CPI-613 at a 50% dose reduction. |
| must be ≤7 days | |

Hematological Adverse Events

| | |
|---|---|
| Grade 1-3 | Maintain dose and schedule. Utilize growth factor and |
| | transfusion supportive care as per institutional guidelines. |
| Grade 4 lasting <7 days | Maintain dose and schedule. Utilize growth factor and |
| | transfusion supportive care as per institutional guidelines. |
| Grade 4 lasting ≥7 days | Hold dose until resolved to grade 3 or less and resume CPI- |
| | 613 at a 50% dose reduction. |

Interruptions of study therapy of greater than 14 days from the expected Cycle 2 Day 1 will result in removal from study. If any subsequent cycle is delayed more than 21 days will result in removal from study.

### Criteria for Therapeutic Response/Outcome Assessment

### Tumor Response

Tumor response will be assessed after Cycle 3 using PET/CT using the RECIL criteria for response assessment in lymphoma (see table below) and/or bone marrow biopsy (depending on sites of disease as indicated by treating physician). For patients with measurable disease, PET response and disease measurements will be made. For patients with only marrow disease not detectable by PET, any development of bone lesions on PET will also be evaluated with a repeat bone marrow biopsy. Should the treating physician determine that disease progression has occurred, the patient will be removed from the study. Should the treating physician determine that the patient has stable disease or has had a favorable response, those patients will continue treatment with CPI-613 for an additional four cycles (approximately twelve weeks). At that time, patients will then undergo re-assessment of disease as above. Patients who are determined by the treating physician to have progressed will be removed from the study, while those who are determined to have stable disease or favorable response will continue, with subsequent re-imaging continuing every 4 cycles for the first year, until disease progression, or patient taken off study.

Assessment of disease response will be the responsibility of the treating physician and should be based on radiologic and/or pathologic findings and performance status.

### Schedule of Response Assessments

For patients with detectable disease by PET/CT scan: PET/CT scan will be performed at baseline, after Cycle 3 of CPI-613 and every 4 cycles of maintenance thereafter until the first year of study treatment.

Patients without detectable disease by PET/CT scan: PET/SC scan will be performed at baseline only, and then as indicated by the treating physician.

Patients with bone marrow involvement: bone marrow biopsy performed at baseline, after Cycle 3 of CPI-613 and every 4 cycles of maintenance thereafter until the first year of study treatment.

Patients without bone marrow involvement: bone marrow biopsy done at baseline, and then as indicated by the treating physician.

RECIL 2017: Criteria for response assessment in lymphoma:

| | Percent change in sum of diameters of target lesions from nadir | | | | |
|---|---|---|---|---|---|
| | Complete | Partial | Minor | Stable | |
| | Response | Response | Response | Disease | Progression of |
| | (CR) | (PR) | (MR) ^{a} | (SD) | Disease (PD) |
| Percent | Complete | ≥30% | ≥10% | <10% | >20% increase |
| change | disappearance | decrease in | decrease in | decrease | in the sum of |
| from | of all | the sum of | the sum of | or ≤20% | longest |
| baseline | target | longest | longest | increase | diameters of |
| | lesions and | diameters of | diameters | in the | target lesions. |
| | all nodes | target | of target | sum of | For small lymph |
| | with long | lesions but | lesions but | longest | nodes measuring <15 |
| | axis <10 | not a CR | not a PR | diameters | mm post |
| | mm. | | (<30%) | of target | therapy, a |
| | ≥30% | | | lesions. | minimum |
| | decrease in | | | | absolute |
| | the sum of | | | | increase of |
| | longest | | | | 5 mm, and the |
| | diameters of | | | | long diameter |
| | target | | | | should exceed |
| | lesions (PR) | | | | 15 mm. |
| | with | | | | Appearance of a |
| | normalization | | | | new lesion |
| | of FDG-PET | | | | |
| FDG-PET | Normalization | Positive | Any | Any | Any |
| | of FDG-PET | (Deauville | | | |
| | (Deauville | score 4-5) | | | |
| | score 1-3) | | | | |
| Bone | Not | Any | Any | Any | Any |
| marrow | involved | | | | |
| involvement | | | | | |
| New | No | No | No | No | Yes or No |
| lesions | | | | | |

FDG-PET, [¹⁸F]2-fluoro-d-deoxy-D-glucose positron emission tomography; CT, computerized tomography
^{a} A provisional category

### Overall Survival

OS will be monitored via office visits and/or telephone contact after treatment termination. OS and PFS will be calculated from the first day of treatment. The duration of OS will be measured until the date of death or censored at follow-up. The duration of response (evaluated by PFS) will be measured from the date a first objective response is documented until the first sign of progression assessed by PET/CT and/or bone marrow biopsy.

### Defining Evaluable Patients

| | |
|---|---|
| Evaluable | Patient received at least 1 dose of CPI-613, taken off study prior |
| for | to 1st response assessment for any reason other than progression |
| Toxicity | of disease. |
| only | Clinical progression or radiographic evidence would be |
| | considered progression of disease and patient would be evaluable |
| | for response. Patients taken off study due to toxicity without a |
| | response assessment (e.g., early treatment-related mortality) will |
| | be considered non-responders. |
| Evaluable for | One of the following options must apply; |
| Toxicity and | Patient received at least 1 dose of CPI-613, came off study for |
| Response | progression of disease. Clinical progression or radiographic |
| | evidence would be considered progression of disease. |
| | Any patient who remained on treatment until first response |
| | assessment per protocol |

Criteria for Removal from Study

Patients will continue on study treatment unless one of the following occurs: Patient exhibits disease progression in the opinion of the treating physician; unacceptable toxicity from CPI-613 occurs per Study Investigator's discretion; if Cycle 2 is more than 14 days delayed from the expected Cycle 2 Day 1; if any subsequent is delayed more than 21 days from Cycle X day 1; patient withdraws consent; treating investigator's discretion to withdraw patients from the study because continued participation in the study is not in the patient's best interest; intercurrent illness such as a condition, injury, or disease unrelated to the intended disease for which the study is investigating, that renders continuing the treatment unsafe or regular follow-up impossible; general or specific changes in the patient's condition that renders the patient ineligible for further investigational treatment; non-compliance with investigational treatment, protocol-required evaluations or follow-up visits; termination of the clinical trial by the study sponsor; death; lost to follow up; pregnancy or a positive pregnancy test.

When terminating treatment during this trial, the investigator should make every effort to contact the patient and to perform a final evaluation. Also, the reason(s) for withdrawal from the study must be recorded.

### Biostatistics

The accrual rate is estimated to be approximately 10 patients/year in a multi-institutional setting, given the rarity of the disease for a total of 34 patients in 3 years. The cohorts will accrue concurrently and be analyzed separately. Cohort 1: patients with relapsed or refractory Burkitt lymphoma/leukemia (n=17). Cohort 2: patients with relapsed or refractory DHL (n=17).

For each cohort: Simon optimal two-stage design (Simon, 1989) will be used for this study. The response rate to any currently single or combination available agent is essentially non-existent without this intervention and any response observed would be beneficial to the patient population. Therefore, we assume the null hypothesis that the current rate is 0.05 (approximately 0) which will be tested against a one-sided alternative of a 25%. Overall response rate. In the first stage, 9 patients will be accrued. If 1 or more participants experience a response by Cycle 3 then the study will continue to the second stage, otherwise the study will be stopped for lack of efficacy. During stage 2, we will accrue an additional 8 patients for a total of 17. If 3 or more responses are observed in the 17 patients, the null hypothesis that the intervention is ineffective will be rejected. This design yields a type I error rate of 0.05 and power of 80%. Additionally, a toxicity interim analysis will be conducted after the first 9 study participants have completed two complete cycles or have gone off study. Should 4 or more of the first 9 patients develop grade 4 toxicity the study will be stopped for toxicity. If 7 or more of the 17 patients developed grade 4 toxicity the treatment will also be considered too toxic for further investigation. This interim analysis has the following operating characteristics for various true toxicity rates.

| | | | | | | |
|---|---|---|---|---|---|---|
| True toxicity rate | 0.20 | 0.26 | 0.32 | 0.38 | 0.44 | 0.50 |
| Probability of declaring | 0.099 | 0.227 | 0.401 | 0.591 | 0.758 | 0.879 |
| the treatment too toxic | | | | | | |

Participants will also be followed and analyzed for secondary outcomes of progression-free and overall survival. Participants will be monitored for survival through routine follow-up visits for up to six months after completion of CPI-613 therapy. Confidence intervals will be calculated around the estimates of the overall response rate (CR, PR, and SD) of CPI-613. We will use Kaplan-Meier method to analyze progression-free and overall survival.

The primary objective of the study is to determine the overall response rate of CPI-613 in patients with relapsed or refractory Burkitt Lymphoma/Leukemia and double hit diffuse large B cell lymphoma analyzed as two separate cohorts. The first assessment is after cycle 3. As per clinical research standard, only patients who reach the first assessment time point are assessable for response. Patients who are off study due to toxicity before the first response assessment will not be replaced, but will be considered non-responders for the purpose of determining the primary endpoint.

Overall Response Rate (ORR) will be defined as rate of complete response (CR)+partial response (PR)+minor response (MR)+Stable disease (SD) as determined as per the RECIL criteria (Younes et. al., “International Working Group consensus response evaluation criteria in lymphoma (RECIL 2017),” Ann Oncol 2017 mdx097. doi: 10.1093/annonc/mdx097). Stable disease will need to be documented on 2 successive assessments.

Duration of response will be calculated for those who have an objective response using Kaplan-Meier method, treating those who are still in response at the end of study as censored.

Progression Free Survival (PFS) will be defined as the time from the date of first cycle until disease progression, withdrawal of consent from active participation in the study, lost to follow-up, death (by any cause) or end of study as censored, whichever is the earliest. PFS will be analyzed by Kaplan Meier method.

Overall Survival (OS) will be defined as the time from date of first cycle and the date of death, withdrawal of consent from active participation in the study, lost to follow-up, or end of study as censored, whichever is the earliest. OS will be analyzed by Kaplan Meier method.

Safety will be assessed by adverse events, physical examinations, vital signs and clinical laboratory tests. All AEs will be monitored and graded using the NCI Common Terminology Criteria for Adverse Events (CTCAE) version 5.0. Descriptive and summary tables of adverse events will be compiled. Adverse events will be coded to system organ class and preferred term using MedDRA.

Exploratory objectives: The response rate will be correlated with pre-treatment biomarkers including variances in IHC and cytokine profiles using Fisher's exact test for categorical markers and Wilcoxon rank sum test for continuous markers. Duration of response, PFS, and OS will be associated with the above biomarkers using Cox proportional hazards model.

Administration of 6,8-bis-benzylthio-octanoic acid

A solution of 6,8-bis-benzylthio-octanoic acid in 1M aqueous triethanolamine is diluted from 50 mg/mL to as low as 4 mg/mL with sterile 5% dextrose for injection (D5W) prior to administration. After dilution with D5W, the solution is clear and has a pH of 8.4-8.8. The D5W solution of 6,8-bis-benzylthio-octanoic acid is administered as an IV infusion over two hours via a central venous catheter on days 1-5 of every cycle.

### Example 2—Treatment of Relapsed or Refractory T-Cell Non-Hodgkin Lymphoma or Classical Hodgkin Lymphoma in Human Patients Using 6,8-Bis-Benzylthio-Octanoic Acid

### Study Design

A phase I clinical trial assessing the safety and efficacy of escalating doses of 6,8-bis-benzylthio-octanoic acid (CPI-613), in combination with bendamustine, in patients with relapsed or refractory T-cell NHL or classic Hodgkin Lymphoma is described. The primary objectives are to determine the MTD of CPI-613, when used in combination with Bendamustine, in patients with relapsed and refractory classic HL or T-cell non-Hodgkin lymphoma and to evaluate the safety of CPI-613+Bendamustine combination. Exploratory objectives are to (a) evaluate Response Rate (RR) and Disease Control Rate (DCR), derived from the modified International Work Group (IWG) criteria and International Cutaneous Lymphoma (Olsen Criteria) for cutaneous lymphomas, (b) evaluate Overall Survival (OS) and Progression-Free-Survival (PFS), and possible correlation between RR and DCR derived from modified IWG criteria vs. OS and PFS, and (c) assess bone marrow biopsy, and possible correlation between Complete Response (CR) vs. bone marrow biopsy assessment (e.g., clear of infiltration of leukemic cells accordingly to morphology, and/or negative on leukemic cells according to immunohistochemistry).

Each treatment cycle is 4 weeks (see table below). CPI-613 at escalating doses is infused intravenously (IV) via a central catheter over 2 hrs on Days 1-4. Bendamustine at 90 mg/m² is infused IV over 10 minutes on Days 4 and 5 of each treatment cycle. On Day 4 when both CPI-613 and Bendamustine are administered, Bendamustine is given immediately after CPI-613 administration. Each patient will be treated with as many as 6 cycles, if clinically indicated.

Patients with limited prior exposure to Bendamustine (less than 2 cycles) will receive a total of 6 cycles of Bendamustine both on and off study. If a patient has had 1 cycle (2 doses) of Bendamustine prior to enrolling on this study, the Bendamustine will be held in Cycle 6. If the patient received 2 cycles of Bendamustine prior to enrolling on this study, the Bendamustine will be held in Cycles 5 and 6.

Timing of CPI-613 and Bendamustine in Each 4-Week Treatment Cycle

| | Day 1 | Day 2 | Day 3 | Day 4 | Day 5 |
|---|---|---|---|---|---|
| Week 1 | CPI-613 | CPI-613 | CPI-613 | CPI-613 | Bendamustine |
| | | | | Bendamustine | |
| Week 2 | | | | | |
| Week 3 | | | | | |
| Week 4 | | | | | |

This study employs a 2-stage dose-escalation scheme to determine the MTD of CPI-613, when used in combination with Bendamustine, in patients with relapsed or refractory T-cell NHL and classic HL, as described below. The assignment of patients will not be randomized, due to the single arm design.

In the single-patient scheme, a single patient will be accrued per dose level. The starting dose of CPI-613 will be 2,000 mg/m², and it will be given in combination with Bendamustine. CPI-613 dose level will be escalated (by an increment of 250 mg/m²) if there is no toxicity attributed as probably or definitely related to CPI-613 or if the toxicity is ≤Grade 1 according to National Cancer Institute (NCI) Common Toxicity Criteria for Adverse Events (CTCAE). If toxicity attributed as probably or definitely related to CPI-613 is ≥Grade 1, the traditional dose-escalation stage will be triggered. Even if there are no toxicities attributed as probably or definitely related to CPI-613 are observed after 2 cohorts (i.e., with the 2,500 mg/m² cohort), the traditional dose-escalation stage will nevertheless be triggered. Therefore, the single patient dose-escalation stage will accrue at most 2 patients.

If DLT occurs in 2 patients at the starting dose of 2,000 mg/m², the dose escalation scheme will be re-started with a revised starting dose of 500 mg/m².

Patients must complete an entire 4-week treatment cycle before the next cohort is initiated. If additional courses of therapy are indicated in a patient, CPI-613 will be dosed identically as in course 1 for that patient. Patients who do not complete a full cycle are not evaluable for MTD, but are still evaluable for toxicities.

All CPI-613 dose escalations conducted in this Traditional Dose-Escalation stage will be with an increment of 250 mg/m². The dose level for CPI-613 of the first cohort in the Traditional Dose-Escalation scheme is the same as that used in the last cohort in the Single-Patient Dose-Escalation scheme. The number of patients in each cohort at this stage will initially be 3, including the first patient in which a >Grade 1 toxicity is observed in the Single-Patient-Dose-Escalation scheme. If no toxicities attributed as probably or definitely related to CPI-613 are observed in the Single-Patient-Dose-Escalation scheme, the starting dose of the Traditional Dose-Escalation stage will be 2,500 mg/m². If no patients in any cohort develop a dose-limiting toxicity (DLT, see definition below), dose escalation will continue in cohorts of 3 patients. However, if a DLT is observed in a patient (whether it is the first, second or third of the 3 intended patients) at any dose level, that cohort will be expanded to a maximum of 6 patients. If no DLT is observed in another patient out of a maximum of 6 patients, dose escalation procedure will continue in 3 patients for each subsequent cohort. However, once a DLT is observed in a total of 2 patients at any dose level, dosing of CPI-613 will stop immediately, even though the total number of patients at the last cohort may be as few as 2. Dose escalation is considered to be complete. The dose level that induces a DLT in 2 or more patients is considered to be above MTD, and the dose level immediately below the dose level that induced a DLT in <2 patients is considered the MTD.

In the event that the DLT in ≥2 patients is not observed once the 3,000 mg/m² cohort has been tested, dose escalation is also considered to be complete since 3,000 mg/m² is the MTD of CPI-613 when given as a single agent. Three additional patients will be enrolled at the MTD for a total of six expected patients at this dose level.

Once the possible MTD dose is determined, this dose arm may be expanded to allow up to 13 additional patients to be enrolled at this dose to determine more information concerning the potential safety of this dose as well as gather preliminary data concerning PFS, OS, RR, DCR etc.

A DLT is defined as any toxicity, at least probably related to CPI-613, which meets the criteria outlined in this paragraph. Events that can clearly be determined to be unrelated to the drug are not considered DLT. The DLT evaluation period is through Cycle 1 (4 weeks) for each patient.

For non-hematological toxicities:
Any non-hematological toxicity Grade≥3, at least possibly related to CPI-613, except for alopecia and nausea uncontrolled by medical management.
Any Grade≥2 toxicity at least possibly related to CPI-613, that does not resolve to Grade≤1 by the start of the next cycle.

For hematologic toxicities:
Grade 4 neutropenia lasting more than 5 days
Febrile neutropenia of any duration (ANC<1.0×10 9/L, fever>38.5° C.)
Grade 4 thrombocytopenia, or Grade 3 thrombocytopenia with bleeding or any requirement for platelet transfusion
Grade 4 anemia, unexplained by underlying disease.

Dose escalation to the next dose level cannot take place until all patients at the previous cohort have been given a complete treatment cycle (i.e., 4 weeks). Intra-patient dose escalation is not allowed in this trial. No patients can participate in more than one cohort.

In the expansion cohort, cycling of CPI-613 and Bendamustine will remain the same as described above. Patients will be administered chemotherapy on days 1-5 of the first week of the cycle, followed by three weeks of no chemotherapy.

Dosing for the expansion cohort will be fixed and will be performed as described in the table below unless otherwise directed by the treating physician.

Expansion Cohort Dosing of CPI-613 and Bendamustine
in Each 4-Week Treatment Cycle

| | Day 1 | Day 2 | Day 3 | Day 4 | Day 5 |
|---|---|---|---|---|---|
| Week 1 | CPI-613: | CPI-613: | CPI-613: | CPI-613: | Bendamustine: |
| | 2500 mg/m² | 2500 mg/m² | 2500 mg/m² | 2500 mg/m² | 90 mg/m² |
| | | | | Bendamustine: | |
| | | | | 90 mg/m² | |
| Week 2 | | | | | |
| Week 3 | | | | | |
| Week 4 | | | | | |

CPI-613 at 2500 mg/m² is infused IV via a central catheter over 2 hrs on Days 1-4. Bendamustine at 90 mg/m² is infused IV over 10 minutes on Days 4 and 5 of each treatment cycle. On Day 4 when both CPI-613 and Bendamustine are administered, Bendamustine is given immediately after CPI-613 administration. Each patient will be treated with as many as 6 cycles, if clinically indicated.

### Dosing Delay and Dose Modification

The occurrence of Grade 1 toxicity does not generally require dose modification for subsequent doses for that patient. However, if Grade 2 non-hematologic toxicity (which includes infectious toxicity) develops and is attributed to at least probably related to CPI-613, treatment can resume only after the Grade 2 toxicity has been reduced to Grade 1 or below, and the dose level for subsequent doses for that patient will be reduced by 25% of the dose at which such Grade 2 toxicity occurs. If Grade 3 or 4 non-hematologic toxicity (which includes infectious toxicity) develops, dosing of that patient will be withheld and the patient shall be monitored for recovery from, and reversibility of, such Grade 3 or 4 toxicity. To resume treatment for a patient who has had Grade 3 or 4 toxicity, the Grade 3 or 4 toxicity must be reduced to Grade 1 or below, and the dose level for subsequent doses for that patient will be reduced to 50% of the dose at which such Grade 3 or 4 toxicity occur. If the reduced dose of CPI-613 results in no toxicity, patients may be dose escalated to the prior dose at the discretion of the treating physician.

For adverse events unrelated to serum creatinine elevation or reduction in renal function but are possibly related to CPI-613, the occurrence of Grade 1 toxicity does not generally require dose modification for subsequent doses for that patient. However, if Grade 2 toxicity (other than nausea) probably related to CPI-613 develops, treatment is to be withheld and can resume only after the Grade 2 toxicity has been reduced to Grade 1 or below, and the dose level for subsequent doses for that patient will be reduced by 25% of the dose at which such Grade 2 toxicity occurs. Grade 2 nausea does not require withholding treatment or dose reduction. If Grade 3 or 4 toxicity probably related to CPI-613 develops, dosing of CPI-613 of that patient will be withheld and the patient shall be monitored for recovery from, and reversibility of, such Grade 3 or 4 toxicity. To resume treatment with CPI-613 for a patient who has had CPI-613-related Grade 3 or 4 toxicity, the Grade 3 or 4 toxicity must be reduced to Grade 1 or below, and the dose level for subsequent doses for that patient will be reduced to 50% of the dose at which such Grade 3 or 4 toxicity occurs.

For adverse events related to creatinine elevation, reduction in renal function or mitochondrial inhibition syndrome that are possibly related to CPI-613, dosing of the patient will be withheld even if the severity level is Grade 1 or above. Treatment can resume only after the toxicity has been reduced to Grade 0. The dose level for subsequent doses for that patient will be reduced by 15% if the severity level is of Grade 1, by 25% for Grade 2 toxicity, and by 50% for Grade 3 or 4 toxicity.

Furthermore, if the toxicity possibly related to CPI-613 is acute renal failure and the severity level is Grade 3 or 4, further patient enrollment will be temporarily suspended in order to enable assessment of the following aspects of the trial and implementation of corrective measures or protocol amendment, if necessary:
compliance of the study sites and investigators to the study protocol
evaluation of the appropriateness of the procedures for monitoring renal function Special note for Mitochondrial Inhibition Syndrome.
This is a constellation of symptoms that may include high fevers, hypotension, lethargy, pancytopenia, altered mental status and generalized weakness and lactic acidosis. If this occurs or is suspected the recommended treatment until symptoms resolve is:
   IV L-carnitine 50 mg/kg/day in divided doses every 4 hours (i.e. 8.3 mg/kg every 4 hours)
   Folic acid 1 mg daily
   Thiamine 100 mg daily

### Dosing Adjustment for Bendamustine Related Toxicities

Administration of Bendamustine should be withheld in the event of ≥Grade 4 hematologic toxicity or clinically significant Grade 2 non-hematologic toxicity. Once non-hematologic toxicity has recovered to Grade 1, Bendamustine can be re-initiated at the discretion of the treating physician. For patients experiencing hematologic toxicity, Bendamustine may be re-initiated at the discretion of the treating physician once counts have improved (Absolute Neutrophil Count [ANC] 1×109/L, platelets 75×109/L). However, if the patient has low blood counts related to underlying disease (i.e., bone marrow involvement or splenic sequestration), the Bendamustine can be re-initiated at the discretion of the treatment physician prior to blood count improvement. At that point, dose reduction as described below should be considered:
Hematologic toxicity Grade 4 toxicity: reduce the dose by 30%.
Non-hematologic toxicity for clinically significant ≥Grade 3 toxicity: reduce the dose by 30%.

Dose re-escalation in subsequent cycles may be considered at the discretion of the treating physician

### Duration of Treatment for Each Patient

Treatment with CPI-613 should be continued as long as the treating physician believes there is clinical benefit, unless or until:
Patients exhibit disease progression
Unacceptable toxicity from CPI-613 in spite of dose reduction
Patient withdrawal of consent
Investigator's discretion to withdraw patients from the study because continued participation in the study is not in the patient's best interest.
Undercurrent illness: a condition, injury, or disease unrelated to the intended disease for which the study is investigating, that renders continuing the treatment unsafe or regular follow-up impossible
General or specific changes in the patient's condition that renders the patient ineligible for further investigational treatment
Non-compliance with investigational treatment, protocol-required evaluations or follow-up visits
Termination of the clinical trial

When terminating treatment during this trial, the investigator should make every effort to contact the patient and to perform a final evaluation. Also, the reason(s) for withdrawal from the study must be recorded.

Upon being taken off the trial, patient's survival and post-study cancer treatment will be monitored by follow up physician visits once patients are removed from trial. All patients will be followed for 5 years post treatment, or until death.

### Patient Inclusion Criteria

Patients must meet all of the following inclusion criteria before enrollment:
1. Histologically or cytologically confirmed T-cell NHL or classic HL (i.e., Nodular Sclerosis HL, Mixed Cellularity HL, Lymphocyte Rich Classic HL, and Lymphocyte Depleted HL) that has relapsed from, or is refractory to, standard therapies (including autologous transplantation) known to provide clinical benefit, but have not been treated with Bendamustine for their lymphoma. Patients with limited exposure to Bendamustine (less than 2 full cycles) may be included, based on PI discretion. Patients with classical Hodgkin Lymphoma must have failed brentuximab vedotin and a PD-1 inhibitor.
2. Must have measurable disease (e.g., a tumor mass>1 cm or evidence of bone marrow involvement).
3. Eastern Cooperative Oncology Group (ECOG) performance status 0-2 (Oken et al., “Toxicity and response criteria of the Eastern Cooperative Oncology Group,” Am J Clin Oncol. 1982; 5(6):649-6551982).
4. Expected survival>3 months.
5. Male and female patients 18 years of age and older.
6. Women of child-bearing potential (i.e., women who are pre-menopausal or not surgically sterile) must use accepted contraceptive methods (abstinence, intrauterine device [IUD], oral contraceptive or double barrier device) during the study, and must have a negative serum or urine pregnancy test within 1 week prior to treatment initiation.
7. Fertile men must practice effective contraceptive methods during the study, unless documentation of infertility exists.
8. At least 2 weeks must have elapsed from any prior surgery.
9. Laboratory values≤2 weeks must be:
   a. Adequate hepatic function (aspartate aminotransferase [AST/SGOT]≤3×upper normal limit [UNL], alanine aminotransferase [ALT/SGPT]≤3×UNL (≤5×UNL if liver metastases present), bilirubin 1.5×UNL).
   b. Adequate renal function (serum creatinine mg/dL or 133 μmon).
   c. Adequate coagulation (“International Normalized Ratio” or INR must be ≤1.5)
10. No evidence of active infection and no serious infection within the past month.
11. Mentally competent, ability to understand and willingness to sign the informed consent form.

### Patient Exclusion Criteria

Patients with the following characteristics are excluded:
1. Known cerebral metastases, central nervous system (CNS) or epidural tumor.
2. History of second malignancy unrelated to HL or NHL and are in complete response and are considered by their physicians to be at less than 30% risk of relapse.
3. Patients receiving any other standard or investigational treatment for their cancer, or any other investigational agent for any indication, within the past 2 weeks prior to initiation of treatment with study drugs.
4. Serious medical illness that would potentially increase patients' risk for toxicity.
5. Any active uncontrolled bleeding, and any patients with a bleeding diathesis (e.g., active peptic ulcer disease).
6. History of abdominal fistula or gastrointestinal perforation ≤6 months prior to treatment with study drugs.
7. Pregnant women, or women of child-bearing potential not using reliable means of contraception (because the teratogenic potential of CPI-613 is unknown).
8. Lactating females.
9. Fertile men unwilling to practice contraceptive methods during the study period.
10. Any condition or abnormality which may, in the opinion of the investigator, compromise the safety of patients.
11. Unwilling or unable to follow protocol requirements.
12. Active heart disease including but not limited to symptomatic congestive heart failure, symptomatic coronary artery disease, symptomatic angina pectoris, symptomatic myocardial infarction or symptomatic congestive heart failure.
13. Patients with a history of myocardial infarction that is <3 months prior to registration.
14. Evidence of active infection, or serious infection within the past month.
15. Patients with known HIV infection, hepatitis B, or hepatitis C.
16. Patients who have received cancer immunotherapy of any type within the past 2 weeks prior to initiation of CPI-613 treatment.
17. Requirement for immediate palliative treatment of any kind including surgery.
18. Albumin<2.0 g/dL or <20 g/L.

### Pretreatment Screening Tests

The following assessments will be done prior to study treatment:

Within 4 weeks prior to Day 1 of study treatment:
tumor assessments, and optional blood and serum samples. (Note: If assessment within this timeframe has already been performed prior to participating in this trial, the results from this assessment can be used.) Within 2 weeks prior to Day 1 of study treatment:
medical history, physical exam, vital signs, height, weight, ECOG, evaluation of symptoms and medications, clinical chemistry, hematology, coagulation, urinalysis, and troponin I.

Within 1 week prior to Day 1 of study treatment:
pregnancy test for women of child-bearing potential.

### Safety Assessment

The safety of CPI-613 and Bendamustine will be assessed based on: evaluation of symptoms; vital signs; ECOG performance status and survival; clinical chemistry (with renal function assessed utilizing the Cockcroft-Gault formula); hematology; coagulation; Troponin I; and ECG.

All safety assessment tests are performed during screening (performed within 2 weeks prior to treatment with CPI-613). Associated with Cycle 1, clinical chemistry, hematology and coagulation are performed within 24 hours prior to dosing CPI-613, and only creatinine results are needed before dosing CPI-613. For Troponin I, it is assessed 1 hour after completion of CPI-613 administration. Vital signs are performed immediately after CPI-613 administration and re-examined only if clinically indicated. For coagulation tests (INR and PTT), they are performed on Day 1 of each cycle. For creatinine and BUN, they are to be checked within 24 hours of every dose of CPI-613. Beyond Cycle 1, physical exam and vital signs, ECOG performance status, evaluation of symptoms and medications are to be performed within 5 days prior to each cycle. Clinical chemistry, hematology, coagulation (INR and PTT), and troponin I will be assessed on Day 1 of each cycle.

### Anti-Tumor Efficacy Assessment

Anti-tumor efficacy is assessed via imaging scans (PET/CT with Dedicated CT (IV contrast)) and bone marrow biopsies performed at baseline, and after every 2 cycles. Additional assessments can be obtained if clinically indicated.

The modified International Wok Group (IWG) criteria (Cheson B D et al., “Revised Response Criteria for Malignant Lymphoma,” J Clin Oncol. 2007; 25(5):579-586) will be used to stage the disease. CR, PR, SD and PD are defined in the table below. RR (the combined rates of CR and PR) and DCR (the combined rates of CR, PR and SD) will also be calculated.

| Response | Definition | Nodal Masses | Spleen, Liver | Bone Marrow |
|---|---|---|---|---|
| CR | Disappearance | (a) FDG-avid or PET positive | Not palpable, | Infiltrate cleared on |
| | of all | prior to therapy; mass of any | nodules | repeat biopsy; if |
| | evidence of | size permitted if PET negative | disappeared | indeterminate by |
| | disease | (b) variably FDG-avid or PET | | morphology, |
| | | negative; regression to normal | | immunhistochemistry |
| | | size on CT | | should be negative |
| PR | Regression | ≥50% decrease in SPD of up | ≥50% decrease in | Irrelevant if |
| | of measureable | to 6 largest dominant masses; | SPD of nodules | positive prior to |
| | disease and | no increase in size of the other | (for single nodule | therapy; cell type |
| | no new sites | nodes | in greatest | should be specified |
| | | (a) FDG-avid or PET positive | transvers | |
| | | prior to therapy; one or more | diameter); no | |
| | | PET positive at previously | increase in size of | |
| | | involved site | liver or spleen | |
| | | (b) variably FDG-avid or PET | | |
| | | negative; regression on CT | | |
| SD | Failure to attain | (a) FDG-avid or PET positive | | |
| | CR/PR or PD | prior to therapy; PET positive | | |
| | | at prior sites of disease and no | | |
| | | new sites on CT or PET | | |
| | | (b) variably FDG-avid or PET | | |
| | | negative; no change in size of | | |
| | | previous lesions on CT | | |
| Relapsed | Any new lesion | Appearance of a new lesion(s) >1.5 | ≥50% increase | New or recurrent |
| disease | or increase | cm in any axis, ≥50% | from nadir in the | involvement |
| or PD | by ≥50% of | increase in SPD of more than | SPD of any | |
| | previously | one node, or ≥50% increase in | previous lesions | |
| | involved sites | longest diameter of a previously | | |
| | from nadir | identified node >1 cm in short axis | | |
| | | Lesions PET positive in FDG-avid | | |
| | | lymphoma or PET positive prior | | |
| | | to therapy | | |

CR, complete remission; FDG, [¹⁸F]2-fluoro-d-deoxy-D-glucose; PET, positron emission tomography; CT, computed tomography; PR, partial remission; SPD, sum of the product of the diameters; SD, stable disease; PD, progressive disease.

The International Society on Cutaneous Lymphomas criteria (Olson E A et al., “Clinical End Points and Response Criteria in Mycosis Fungoides and Sézary Syndrome: A Consensus Statement of the International Society for Cutaneous Lymphomas, the United States Cutaneous Lymphoma Consortium, and the Cutaneous Lymphoma Task Force of the European Organisation for Research and Treatment of Cancer,” J Clin Oncol. 2011; 29(18):2598-2607) will be used to stage the disease of cutaneous lymphoma patients. CR, NI, PR, PD and SD are defined as shown below. Also shown are the response criteria for each component of the TNMB staging (i.e., skin, nodes, viscera, and blood) which will be used to define the global response (GR).

Global Response Score

| | | | | | |
|---|---|---|---|---|---|
| Global | | | | | |
| Score* | Definition | Skin | Nodes | Blood | Viscera |

| | | | |
|---|---|---|---|
| CR | Complete | CR | All categories have CR/NI |
| | disappearance of all | | |
| | clinical evidence of | | |
| | disease | | |
| PR | Regression of | CR | All categories do not have a CR/NI and |
| | measurable disease | | no category has a PD |
| | | PR | No category has a PD and if any |
| | | | category involved at baseline, at least |
| | | | one has a CR or PR |
| SD | Failure to attain CR, PR, | PR | No category has a PD and if any |
| | or PD representative of | | category involved at baseline, no CR or |
| | all disease | | PR in any |
| | | SD | CR/NI, PR, SD in any category and no |
| | | | category has a PD |
| PD | Progressive disease | | PD in any category |
| Relapse | Recurrence disease in | | Relapse in any category |
| | prior CR | | |

*Abbreviations: CR, complete response; NI, noninvolved; PR, partial response; PD, progressive disease; SD, stable disease.

Response in Skin

| Response | Definition |
|---|---|
| Complete | 100% clearance of skin lesions |
| response | |
| Partial | 50%-99% clearance of skin disease from baseline without new |
| response | tumors (T₃) in patients with T₁, T₂, or T₄ only skin |
| Stable disease | <25% increase to < 50% clearance in skin disease from baseline |
| | without new tumors (T₃) in patients with T₁, T₂, or T₄ only skin |
| | disease |
| Progressive | ≥25% increase in skin disease from baseline or |
| disease† | |
| | New tumors (T₃) in patients with T₁, T₂, or T₄ only skin disease or |
| | Loss of response in those with complete or partial response, |
| | increase of skin score of greater than the sum of nadir plus 50% |
| | baseline score |
| Relapse | Any disease recurrence in those with complete response |

Response in Lymph Nodes*

| Response | Definition |
|---|---|
| CR | All lymph nodes are now ≤1.5 cm in greatest transverse (long axis) |
| | diameter by method used to assess lymph nodes at baseline or biopsy |
| | negative for lymphoma; in addition, lymph nodes that were N₃ |
| | classification and ≤1.5 cm in their long axis and >1 cm in their short |
| | axis at baseline, must now be ≤1 cm in their short axis or biopsy |
| | negative for lymphoma |
| PR | Cumulative reduction ≥50% of the SPD of each abnormal lymph node |
| | at baseline and no new lymph node >1.5 cm in the diameter of the |
| | long axis or >1.0 cm in the diameter of the short axis if the long axis is |
| | 1-1.5 cm diameter |
| SD | Fails to attain the criteria for CR, PR, and PD |
| PD† | ≥50% increase in SPD from baseline of lymph nodes or |
| | Any new node > 1.5 cm in the long axis or > 1 cm in the short axis if |
| | 1-1.5 cm in the long axis that is proven to N₃ histologically or |
| | Loss of response; >50% increase from nadir in SPD of lymph nodes in |
| | those with PR |
| Relapse | Any new lymph node >1.5 cm in the long axis in those with CR proven |
| | to be N₃ histologically |

Response in Viscera

| Response | Definition |
|---|---|
| CR | Liver or spleen or any organ considered involved at baseline should not |
| | be enlarged on physical exam and should be considered normal by |
| | imaging; no nodules should be present on imaging of liver or spleen; |
| | any post treatment mass must be determined by biopsy to be negative |
| | for lymphoma |
| PR | ≥50% regression in any splenic or liver nodules, or in measurable |
| | disease (SPD) in any organs abnormal at baseline; no increase in size |
| | of liver or spleen and no new sites of involvement |
| SD | Fails to attain the criteria for CR, PR, or PD |
| PD* | >50% increase in size (SPD) of any organs involved at baseline or |
| | New organ involvement or |
| | Loss of response: >50% increase from nadir in the size (SPD) of any |
| | previous organ involvement in those with PR |
| Relapse | New organ involvement in those with CR |

Response in Blood*

| Response | Definition |
|---|---|
| CR† | B₃ |
| PR‡ | >50% decrease in quantitative measurements of blood tumor burden |
| | from baseline in those with high tumor burden at baseline (B₂) |
| SD | Fails to attain criteria for CR, PR, or PD |
| PD§ | B₃ to B₂ or |
| | >50% increase from baseline and at least 5,000 neoplastic cells/μL [symbol] or |
| | Loss of response: in those with PR who were originally B₂ at baseline, |
| | >50% increase from nadir and at least 5,000 neoplastic cells/μL |
| Relapse | Increase of neoplastic blood lymphocytes to ≥ B₁ in those with CR |

[symbol]

indicates data missing or illegible when filed

Survival (and information related to cancer treatment received after the study and disease status) will be obtained bimonthly via telephone contact after treatment termination. Medical record review will also be performed to coincide with the bimonthly telephone call to obtain information regarding evidence and of relapse (as documented on CT, PET, biopsy, etc).

OS will be determined based on the time from the first dose of CPI-613 to death by any cause. PFS will be determined based on the time from the first dose of CPI-613 to DP.

### Specifics of Tests Performed During the Study

The ECOG Performance Status scales (Oken M M et al., “Toxicity and response criteria of the Eastern Cooperative Oncology Group,” Am J Clin Oncol. 1982; 5(6):649-655) will be used to assess how a patient's disease is progressing and assess how the disease affects the daily living abilities of the patient. These scales are listed below. The higher the ECOG score, the worse the prognosis.

| Grade | ECOG |
|---|---|
| 0 | Fully active, able to carry on all pre-disease performance without restriction |
| 1 | Restricted in physically strenuous activity but ambulatory and able to carry out work of a |
| | light or sedentary nature, e.g., light house work, office work |
| 2 | Ambulatory and capable of all self-care but unable to carry out any work activities. Up and |
| | about more than 50% of waking hours |
| 3 | Capable of only limited self-care, confined to bed or chair more than 50% of waking hours |
| 4 | Completely disabled. Cannot carry on any self-care. Totally confined to bed or chair |
| 5 | Dead |

Clinical chemistry assessed includes: glucose; BUN; creatinine; AST/serum glutamic-oxaloacetic transaminase (S GOT); total protein; ALT/serum glutamic-pyruvic transaminase (SGPT); albumin; alkaline phosphatase (ALP); Na+; K+; total bilirubin; Cl−; Mg; Ca+2; PO4; and CO2.

Hematology includes: complete blood count; hemoglobin; differential count; hematocrit; and platelet count. Coagulation includes INR and partial thromboplastin time. Cardiac Safety includes Troponin I and ECG.

### Study Drugs

Bendamustine at 90 mg/m2 is infused IV over 10 minutes on Days 4 and 5 of each treatment cycle. On Day 4 when both CPI-613 and Bendamustine are administered, Bendamustine is given immediately after CPI-613 administration.

CPI-613 is to be given as 2-hr IV infusion via a central venous catheter. The dose of CPI-613 is in a dose-escalation manner.

CPI-613 is slightly photosensitive and is provided in 10-mL amber glass vials. Each vial contains 10 mL of CPI-613 at a concentration 50 mg/mL, equivalent to 500 mg of CPI-613. The drug product of CPI-613 is a clear and colorless solution that is free of any particulate matter. CPI-613 should be stored under refrigeration, at 2°-8° C. (36°-46° F.), except when being prepared for administration.

CPI-613 must be administered IV by infusion, via an IV catheter with D5W running at a rate of about 125-150 mL/hr. To avoid local reactions at and around the site of administration, CPI-613 should be administered via a central venous catheter.

CPI-613 can cause leaching of Diethylhexyl Phthalate (DEHP) from IV infusion sets and IV bags (Study COM-003). Therefore, DEHP-containing IV infusion sets, IV bags or syringes should not be used in mixing or administration of CPI-613. Examples of the IV sets, IV bags and syringes that do not contains DEHP and therefore can be used in the administration of CPI-613 are:
Extension Set for Syringe Pump Use: All extension sets from MED-RX do not contain DEHP.
Syringes: Kendall Monoject syringes, all mono-ject syringes are DEHP free.
   A compatibility study has been conducted showing that CPI-613 is compatible with 4 commonly used IV infusion sets. Therefore, these 4 types of IV infusion sets, and IV infusion sets that are made with the same materials, can be used to administer CPI-613. These IV infusion sets are:
PVC material—ADDitIVâ Primary IV Set with Universal Spike, Backcheck Valve, 2 Injection Sites, DEHP-Free and Latex-Free, 15 drops/mL, REF V14453, B Braun Medical Inc.
Latex material—Interlinka System Secondary Medication Set, 10 drops/mL, 2C7451, Baxter Healthcare Corporation
PVC material—Surshield™ Safety Winged Infusion Set, 0.19 mL Volume, Latex-Free, DEHP-Free, SV*S25BLS, Terumo Medical Products Hangzhou Co. Ltd.
Polyethylene material—Interlinka System Paclitaxel Set by Baxter HealthCare, Non
DEHP-free: Polyethylene tubing with a 0.22 microfilter Item #2C7558 10 drops/mL Compatibility studies have shown that CPI-613 drug product (50 mg/mL), and drug product diluted with D5W to various concentrations (1.6-25 mg/mL) are compatible with various types of syringes, as listed below. Therefore, any of these types of syringes, and syringes that are made with the same materials, can be used to administer CPI-613. Also, glass syringes can also be used, since glass (such as glass containers) is compatible with CPI-613 drug product.
Norm-Ject, polyethlyene barrel, polyethylene plunger, latex free (Henke Sass Wolf GMBH) syringes
Becton Dickinson syringes
Terumo syringes
Monoject syringes
Glass syringes

CPI-613 must be diluted from 50 mg/mL to 12.5 mg/mL with 5% Dextrose Water (D5W) (i.e., 1 portion of CPI-613 diluted with 3 portions of D5W) prior to administration. The diluted drug product should be visually inspected for clarity. If haziness, precipitate or coloration (other than colorless) is observed, do not use the diluted drug product for dosing. After dilution with sterile D5W, the solution is clear and has a pH of 8.4-8.8. The diluted CPI-613 drug product has been found to be stable for 24 hrs at room temperature and refrigeration temperature.

CPI-613 must be administered IV, via an IV catheter that is free flowing and free of air in the dead space of the IV catheter, to minimize vascular irritation, inflammation and acute toxicity of CPI-613. Accidental co-administration of extra air in the dead space of IV catheters during administration of CPI-613 has demonstrated the potential to induce acute toxicity of CPI-613 according to animal studies. Also, accidental leakage of CPI-613 into the perivascular space during IV administration, which prolongs exposure of perivascular tissue to CPI-613, can induce significant local inflammation according to animal studies. To avoid local reactions at and around the site of administration, CPI-613 must be administered via a central venous catheter.

CPI-613 must not be administered as a bolus, but by infusion, at a rate of ˜0.5 mL/min, via a central venous catheter with D5W running at a rate of about 125-150 mL/hr. This is to minimize potential acute toxicity of CPI-613, according to animal studies.

The following precautions must be taken when administering CPI-613: Confirmation of the placement of the IV line to ensure a lack of leakage of CPI-613 into the perivascular space; confirmation that the IV line is free flowing; confirmation that the IV line is free of dead air space; dilute CPI-613 drug product with D5W, as instructed in the study protocol; administer CPI-613 by infusion, not as a bolus; after administration of CPI-613, flush the IV line with ˜10 mL of D5W to remove residual CPI-613; and to avoid local reactions at and around the site of administration, CPI-613 should be administered via a central venous catheter.

The amount of CPI-613 at each dose level is based on the BSA of the patient. The BSA values will be calculated based on the height and body weight taken during screening and this BSA value is used throughout the study. This is unless there is a >10% change in the body weight from baseline during the study. At that point, BSA should be revised based on the new body weight and height. The new BSA values will be used from that point on for the remainder of the study, unless there is another >10% change in body weight which will require another revision of the BSA.

Patients cannot receive any standard or investigational treatment (except CPI-613 and Bendamustine) for their cancer, or any other investigational drugs for any indications, while on this study. All concomitant medications (including trade and generic names, dosage and dosing schedule) must be recorded. Concomitant use of anti-emetics is permitted for patients with disease-related nausea. If study subjects experience metallic taste/taste alteration during CPI-613 infusion, which can sometimes lead to nausea and vomiting, patients can take mint candy to minimize the adverse effect. The use of mint candy, and its effectiveness in minimizing metallic taste/taste alteration, should also be recorded.

Special note for Mitochondrial Inhibition Syndrome. This is a constellation of symptoms that may include high fevers, hypotension, lethargy, pancytopenia, altered mental status and generalized weakness and lactic acidosis. If this occurs or is suspected the recommended treatment until symptoms resolve is:
IV L-carnitine 50 mg/kg/day in divided doses every 4 hours (i.e. 8.3 mg/kg every 4 hours)
Folic acid 1 mg daily
Thiamine 100 mg daily

For Bendamustine, prophylactic treatment for drug-related symptoms can be given according to Package Inserts of the study drugs and clinical practice. Supportive treatment may include anti-emetic, anti-diarrhea, anti-pyretic, anti-allergic, anti-hypertensive, analgesics, antibiotics, allopurinol, and others such as blood products and bone marrow growth factors. Patients may use erythropoietin for chronic anemia. The investigator may utilize erythropoietic factors, or blood or platelet transfusions at their discretion.

### Adverse Events

A DLT is defined as follows:
For non-hematological toxicities:
   Any non-hematological toxicity Grade≥3, except for alopecia and nausea uncontrolled by medical management.
   Any Grade≥2 toxicity that does not resolve to Grade≤1 by the start of the next cycle.
For hematologic toxicities:
   Grade 4 neutropenia lasting more than 5 days
   Febrile neutropenia of any duration (ANC<1.0×10 9/L, fever>38.5° C.)
   Grade 4 thrombocytopenia, or Grade 3 thrombocytopenia with bleeding or any requirement for platelet transfusion
   Grade 4 anemia, unexplained by underlying disease

The DLT evaluation period is through Cycle 1 (4 weeks) for each patient. NOTE: Toxicities must continue to be collected during Cycles 2, 3, 4, 5 and 6 for data analysis purposes.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating a lymphoma, comprising the step of administering to a patient in need thereof a therapeutically effective amount of 6,8-bis-benzylthio-octanoic acid or a pharmaceutically acceptable salt thereof pursuant to a treatment cycle of at least two weeks, wherein during each treatment cycle the 6,8-bis-benzylthio-octanoic acid or a pharmaceutically acceptable salt thereof is administered during the first week but is not administered after the first week, and the 6,8-bis-benzylthio-octanoic acid or a pharmaceutically acceptable salt thereof is administered at a dose of about 2,500 mg/m² or less on each day that it is administered, in order to treat the lymphoma.

2. The method of claim 1, wherein the lymphoma is Stage I.

3. The method of claim 1, wherein the lymphoma is Stage II.

4. The method of claim 1, wherein the lymphoma is Stage III.

5. The method of claim 1, wherein the lymphoma is Stage IV.

6. The method of any one of claims 1-5, wherein the lymphoma is a T-cell lymphoma.

7. The method of any one of claims 1-5, wherein the lymphoma is a B-cell lymphoma.

8. The method of any one of claims 1-5, wherein the lymphoma is relapsed or refractory Burkitt's lymphoma.

9. The method of any one of claims 1-5, wherein the lymphoma is double hit diffuse large B cell lymphoma.

10. The method of any one of claim 8 or 9, wherein the treatment cycle comprises an induction phase followed by a maintenance phase, wherein the induction phase comprises two, two-week cycles and the maintenance phase comprises one or more three-week cycles, and the 6,8-bis-benzylthio-octanoic acid or pharmaceutically acceptable salt thereof is administered on days 1-5 of each cycle.

11. The method of claim 10, wherein the 6,8-bis-benzylthio-octanoic acid or pharmaceutically acceptable salt thereof is administered at a daily dosage of about 2,500 mg/m² on days 1-5 of each cycle.

12. The method of any one of claims 1-5, wherein the lymphoma is relapsed or refractory Hodgkin lymphoma.

13. The method of claim 12, wherein the patient has failed brentuximab vedotin and a PD-1 inhibitor.

14. The method of any one of claims 1-5, wherein the lymphoma is relapsed or refractory T-cell non-Hodgkin lymphoma.

15. The method of any one of claims 12-14, wherein the treatment cycle is four weeks and the 6,8-bis-benzylthio-octanoic acid or pharmaceutically acceptable salt thereof is administered on days 1-4 of each cycle.

16. The method of claim 15, further comprising the step of administering to the patient a therapeutically effective amount of bendamustine hydrochloride.

17. The method of claim 16, wherein the bendamustine hydrochloride is administered at a daily dosage of about 90 mg/m² on days 4 and 5 of each cycle.

18. A method for treating relapsed or refractory Burkitt's lymphoma, comprising the step of administering to a patient in need thereof 6,8-bis-benzylthio-octanoic acid or a pharmaceutically acceptable salt thereof pursuant to a treatment regimen comprising two 14-day induction cycles followed by one or more 21-day maintenance cycles, wherein the 6,8-bis-benzylthio-octanoic acid or a pharmaceutically acceptable salt thereof is administered as a single daily dose of about 2,500 mg/m² on each of days 1, 2, 3, 4, and 5 of each cycle and is not administered on other days of the cycle, in order to treat the relapsed or refractory Burkitt's lymphoma.

19. A method for treating high-grade B-cell lymphoma with rearrangements of MYC and BCL2 and/or BCL6, comprising the step of administering to a patient in need thereof 6,8-bis-benzylthio-octanoic acid or a pharmaceutically acceptable salt thereof pursuant to a treatment regimen comprising two 14-day induction cycles followed by one or more 21-day maintenance cycles, wherein the 6,8-bis-benzylthio-octanoic acid or a pharmaceutically acceptable salt thereof is administered as a single daily dose of about 2,500 mg/m² on each of days 1, 2, 3, 4, and 5 of each cycle and is not administered on other days of the cycle, in order to treat the high-grade B-cell lymphoma with rearrangements of MYC and BCL2 and/or BCL6.

20. The method of claim 18 or 19, wherein the treatment regimen comprises at least 5 maintenance cycles.

21. A method for treating relapsed or refractory classic Hodgkin lymphoma, comprising the step of administering to a patient in need thereof a therapeutically effective amount of
a. 6,8-bis-benzylthio-octanoic acid or a pharmaceutically acceptable salt thereof, and
b. bendamustine hydrochloride;
pursuant to a treatment cycle of four weeks, wherein the 6,8-bis-benzylthio-octanoic acid or a pharmaceutically acceptable salt thereof is administered only on days 1, 2, 3, and 4 of each treatment cycle and the bendamustine hydrochloride is administered only on days 4 and 5 of each treatment cycle, and the 6,8-bis-benzylthio-octanoic acid or a pharmaceutically acceptable salt thereof is administered as a single dose of about 2,500 mg/m² on each day that it is administered and the bendamustine hydrochloride is administered as a single dose of about 90 mg/m² on each day that it is administered, in order to treat the relapsed or refractory classic Hodgkin lymphoma.

22. The method of claim 21, wherein the patient has failed brentuximab vedotin and a PD-1 inhibitor.

23. A method for treating relapsed or refractory T-cell non-Hodgkin lymphoma, comprising administering to a patient in need thereof a therapeutically effective amount of
a. 6,8-bis-benzylthio-octanoic acid or a pharmaceutically acceptable salt thereof, and
b. bendamustine hydrochloride;
pursuant to a treatment cycle of four weeks, wherein the 6,8-bis-benzylthio-octanoic acid or a pharmaceutically acceptable salt thereof is administered only on days 1, 2, 3, and 4 of each treatment cycle and the bendamustine hydrochloride is administered only on days 4 and 5 of each treatment cycle, and the 6,8-bis-benzylthio-octanoic acid or a pharmaceutically acceptable salt thereof is administered as a single dose of about 2,500 mg/m² on each day that it is administered and the bendamustine hydrochloride is administered as a single dose of about 90 mg/m² on each day that it is administered, in order to treat the relapsed or refractory T-cell non-Hodgkin lymphoma.

24. The method of any preceding claim, wherein the 6,8-bis-benzylthio-octanoic acid or a pharmaceutically acceptable salt thereof comprises an ion pair of 6,8-bis-benzylthio-octanoic acid with triethanolamine.

25. The method of claim 24, wherein the 6,8-bis-benzylthio-octanoic acid or pharmaceutically acceptable salt thereof is formulated as 50 mg/mL solution of 6,8-bis-benzylthio-octanoic acid in 1 M (150 mg/mL) aqueous triethanolamine.

26. The method of claim 24 or 25, wherein the ion pair of 6,8-bis-benzylthio-octanoic acid with triethanolamine is administered intravenously to the patient.

27. The method of claim 25, wherein the solution of 6,8-bis-benzylthio-octanoic acid in 1 M (150 mg/mL) aqueous triethanolamine is diluted from 50 mg/mL to as low as 4 mg/mL with sterile 5% dextrose for injection (D5W), and then the diluted solution is administered to the patient as an IV infusion over two hours via a central venous catheter.

28. The method of claim 27, wherein the 50 mg/mL solution is diluted with D5W to a 6,8-bis-benzylthio-octanoic acid concentration of 12.5 mg/mL, and then the diluted solution is administered to the patient as an IV infusion over two hours via a central venous catheter.

29. A medical kit, comprising (i) 6,8-bis-benzylthio-octanoic acid or a pharmaceutically acceptable salt thereof, and (ii) instructions for treating a lymphoma in a patient according to the method of any preceding claim.
